SEQUENCE LISTING (33 sequences)

SEQ ID NO: 1 - protein, 449 aa, Borrelia burgdorferi
MPPKVKIKNDFEIFRKELEILYKKYLNNELSYLKLKEKLKILAENHKAILFRKDKFTNRSIILNLSKTRKIIKEYINLSVIERIRRDNTFLFFWKSRRIKELKNIGIKDRKKIEELIFSNQMNDEKSYFQYFIDLFVTPKWLNDYAHKYKIEKINSYRKEQIFVKINLNTYIEIIKLLLNQSRDIRLKFYGVLMAIGRRPVEVMKLSQFYIADKNHIRMEFIAKKRENNIVNEVVFPVFADPELIINSIKEIRYMEQTENLTKEIISSNLAYSYNRLFRQIFNNIFAPEESVYFCRAIYCKFSYLAFAPKNMEMNYWITKVLGHEPNDITTAFHYNRYVLDNLDDKADNSLLTLLNQRIYTYVRRKATYSTLTMDRLESLIKEHHIFDDNYIKTLIVIKNLMLKDNLETLAMVRGLNVKIRKAFKATYGYNYNYIKLTEYLSIIFNYKL

SEQ ID NO: 2 - DNA, 70 bp, Borrelia burgdorferi
gagtcaaaatactctatactaataaaaaattatatatataattttttattagtatagagtattttgactc

SEQ ID NO: 3 - DNA, 50 bp, Borrelia burgdorferi
actctatactaataaaaaattatatatataattttttattagtatagagt

SEQ ID NO: 4 - DNA, 38 bp, Borreilia burgdorferi
tactaataaaaaattatatatataattttttattagta

SEQ ID NO: 5 - DNA, 35 bp, Borrelia burgdorferi
atataattttttattagtatagagtattttgactc

SEQ ID NO: 6 - DNA, 25 bp, Borrelia  burgdorferi
atataattttttattagtatagagt

SEQ ID NO: 7 - DNA, 19 bp, Borrelia burgdorferi
atataattttttattagta

SEQ ID NO: 8 - DNA, 27 bp, Borrelia burgdorferi
atataattttttattagtatagagtat

SEQ ID NO: 9 - DNA, 18 bp, Borrelia burgdorferi
atataattttttattagt

SEQ ID NO: 10 - DNA, 16 bp, Borrelia burgdorferi
atataatttttttatt

SEQ ID NO: 11 - DNA, 31 bp, Borrelia burgdorferi
tattaaatataattttttattagtatagagt

SEQ ID NO: 12 - DNA, 28 bp, Borrelia burgdorferi
taaatataattttttattagtatagagt

SEQ ID NO: 13 - DNA, 27 bp, Borrelia burgdorferi
aaatataattttttattagtatagagt

SEQ ID NO: 14 - DNA, 25 bp, Borrelia burgdorferi
taaatataattttttagtatagagt

SEQ ID NO: 15 - DNA, 22 bp, Borrelia burgdorferi
atataattttttagtatagagt

SEQ ID NO: 16 - DNA, 24 bp, Borrelia burgdorferi
atataattttttatagtatagagt

SEQ ID NO: 17 - DNA, 25 bp, Borrelia burgdorferi
atataattttttattaatagatccg

SEQ ID NO: 18 - DNA, 25 bp, Borrelia burgdorferi
atataattttttattaggatccact

SEQ ID NO: 19 - protein, 23 aa, Artificial Sequence
  Tn5
ETPLKWLYTHRWRIEEFHKAWKT

SEQ ID NO: 20 - protein, 21 aa, Artificial Sequence
  Tn10
KEPWIYSKRMQIEETFRDLKS

SEQ ID NO: 21 - protein, 24 aa, Borrelia burgdorferi
VTPKWLNDYAHKYKIEKINSYRKE

SEQ ID NO: 22 - protein, 25 aa, Artificial Sequence
  Box A
FYGVLMAIGRRPVEVMKLSQFYIAD

SEQ ID NO: 23 - protein, 20 aa, Artificial Sequence
  Box C
ITKVLGHEPNDITTAFHYNR

SEQ ID NO: 24 - protein, 6 aa, Artificial Sequence
  Peptide #2
WRRCRW

SEQ ID NO: 25 - protein, 6 aa, Artificial Sequence
  Peptide #5
WRRWCR

SEQ ID NO: 26 - protein, 6 aa, Artificial Sequence
  Peptide #7
WRYRCR

SEQ ID NO: 27 - protein, 6 aa, Artificial Sequence
  Peptide #8
WRWYCR

SEQ ID NO: 28 - protein, 6 aa, Artificial Sequence
  Peptide #10
RCCYWW

SEQ ID NO: 29 - protein, 6 aa, Artificial Sequence
  Peptide #52
WKHYNY

SEQ ID NO: 30 - protein, 6 aa, Artificial Sequence
  Peptide #59
KWWCRW

SEQ ID NO: 31 - protein, 8 aa, Artificial Sequence
  Peptide
WRWYCRCK

SEQ ID NO: 32 - DNA, 43 bp, Artificial Sequence
  B53 primer
aatacgttgagggtctcacatgcctccaaaagtgaagataaaa

SEQ ID NO: 33 - DNA, 45 bp, Artificial Sequence
  B52 primer
gtgcccggatccctatagcttataattaaaaattattgataagta

(19) United States
(12) Patent Application Publication
(10) Pub. No.: US 2003/0170693 A1
(43) Pub. Date: Sep. 11, 2003

(54) Assay for identifying modulators of Borrelia telomere resolvase

(76) Inventors: George Chaconas, Calgary (CA); Kerri Kobryn, London (CA); Yvonne M. Tourand, Calgary (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/326,587

(22) Filed: Dec. 20, 2002

Related U.S. Application Data
(60) Provisional application No. 60/341,752, filed on Dec. 21, 2001.

(51) Int. Cl. C12Q 1/68; A61K 48/00

(52) U.S. Cl. 435/6; 514/44

(57) ABSTRACT

The present invention relates to a method of identifying modulators of Borrelia telomere resolvase. The method involves incubating the Borrelia telomere resolvase enzyme, ResT, in the presence of a test substance and a telomere resolution substrate comprising a functional replicated telomere. Substances identified using the method of the invention may be potent inhibitors of Borrelia infection.

This application claims the benefit under 35 USC §119(e) from U.S. Provisional patent application serial No. 60/341,752, filed Dec. 21, 2001.

### FIELD OF THE INVENTION

The present invention relates generally to protein assays. More specifically, the present invention relates to assays for identifying modulators of the telomere resolvase enzyme involved in DNA replication in Borrelia.

### BACKGROUND OF THE INVENTION

The problem of replicating the 3′ ends of linear template DNA molecules was first described in the 1970's (Watson, 1972). In most bacteria this problem has been circumvented through the use of circular chromosomes, while in eukaryotic organisms the problem has been solved by telomerase-mediated extension of DNA ends. However, alternative approaches to solving this problem do exist in nature (see Casjens, 1999; Hinnebusch and Tilly, 1993; Kobryn and Chaconas, 2001; Kornberg, 1992; Meinhardt et al., 1997; Nosek et al., 1998; Rybchin and Svarchevsky, 1999; Traktman, 1996; Volff and Altenbuchner, 2000)). One such strategy employed by a wide variety of organisms (poxviruses, African Swine Fever Virus, Chlorella virus, certain yeast mitochondrial plasmids, the E. coli phage N15 and bacteria in the genus Borrelia) is the use of covalently closed hairpin “telomeres” at the ends of their linear DNA molecules. In the three systems where studies of the replication process for linear molecules with hairpin ends have been described (poxviruses, E. coli phage N15 and B. burgdorferi), the use of a “telomere resolution” step has been reported (see (Kobryn and Chaconas, 2001) for a recent review). Telomere resolution is a DNA breakage and reunion reaction used to process replication intermediates and to regenerate covalently closed hairpin telomeres.

The genus Borrelia contains spirochetes causing Lyme disease and relapsing fever (Barthold, 2000; Nordstrand et al., 2000; Schwan et al., 1999; Shapiro and Gerber, 2000) and is the only known bacterial genus characterized by linear replicons containing covalently closed hairpin ends (Barbour and Garon, 1987; Casjens et al., 1997; Hinnebusch and Barbour, 1991). The genome of Borrelia burgdorferi B31, the prototype agent for Lyme disease, has a segmented genome with a linear chromosome of 911 kb as well as at least 12 linear and nine circular extrachromosomal elements (Casjens et al., 2000; Fraser et al., 1997). The linear replicons all contain inverted repeat hairpin telomeres, and the mechanism by which such molecules are replicated in B. burgdorferi has been a subject for speculation (Casjens, 1999; Hinnebusch and Tilly, 1993; Marconi et al., 1996). Recent work in B. burgdorferi has supported the replication strategy that is presented in FIG. 1A. Picardeau and coworkers mapped a bidirectional origin of replication to a 2 kb region in the center of the 911 kb chromosome using nascent DNA strand analysis (Picardeau et al., 1999). Using CG-skew analysis they also predicted that the 12 linear extra-chromosomal elements have internal bidirectional origins (Picardeau et al., 2000). Internal initiation, as opposed to initiation at the telomeres, implies a circular replication intermediate. Such an intermediate (not yet observed in vivo) would require a DNA breakage and reunion event (telomere resolution) to reconstitute linear replicons with hairpin telomeres. The existence of such a step in Borrelia DNA replication was recently suggested (Chaconas et al., 2001). Synthetic 140 bp and 70 bp replicated telomeres (L′-L shown in FIG. 1A) corresponding to the left end telomere of Ip17 were shown to function as viable substrates for telomere resolution in vivo. The replicated B. burgdorferi telomeres were sufficient for telomere resolution at an internal site in Ip17 and were also able to convert a cp9 derived circular plasmid into a linear replicon.

Telomere resolution can theoretically occur by either of two pathways. One involves the use of a Holliday junction resolvase mechanism and the other a topoisomerase-like mechanism with a covalent protein-DNA intermediate (see (Kobryn and Chaconas, 2001)). Interestingly, B. burgdorferi encodes at least 10 versions of a putative Holliday junction resolving enzyme (Aravind et al., 2000) which are possible candidates for the telomere resolvase. In addition, a protein encoded by the BBB03 locus, with limited sequence homology (Rybchin and Svarchevsky, 1999) to the recently discovered telomere resolvase from the E. coli phage N15 (Deneke et al., 2000), is another possible candidate for the telomere resolution activity of B. burgdorferi.

In view of the foregoing, there is a need to positively identify the gene and corresponding protein involved in telomere resolution in Borrelia. Once such a gene and protein are identified, they may be used to develop assays for substances that modulate telomere resolution, and therefore DNA replication in Borrelia and other organisms in which telomere resolution occurs via a telomere resolvase mechanism. Such substances may be useful as effective agents against, for example, Borrelia infection.

### SUMMARY OF THE INVENTION

The present inventors were the first to identify the gene which encodes the protein responsible for telomere resolution in Borrelia burgdorferi by unequivocally showing that the BBB03 locus, carried by the circular plasmid cp26, encodes the B. burgdorferi telomere resolvase (Kobryn and Chaconas, 2002). This protein is referred to herein as ResT, for Resolvase of telomeres. Accordingly, the present invention provides an isolated ResT protein that is useful as a telomere resolvase. ResT is a highly efficient telomere resolvase in a reaction that does not require accessory proteins, divalent metal ions or a high energy cofactor.

Since ResT is responsible for telomere resolution, modulators of the telomere resolution reaction promoted by ResT may modulate telomere resolution in all organisms for which a telomere resolvase is involved in the replication of linear replicons. Accordingly, the present invention provides a method of modulating telomere resolution comprising administering an effective amount of a modulator of ResT to a cell or animal in need thereof.

Telomere resolution by ResT has been confirmed in the genus Borrelia, therefore inhibitors of the telomere resolution reaction promoted by ResT should block DNA replication of all linear replicons in Borrelia species and hence act as highly effective anti-borrelial agents. Accordingly, the present invention provides a method of treating or preventing Borrelia infection comprising administering an effective amount of an inhibitor of ResT to an animal in need thereof.

The present invention also relates to the use of the ResT protein in assays for identifying substances that modulate telomere resolution. The activity of the purified recombinant (Kobryn and Chaconas, 2002) protein can be assayed using nucleic acid sequences, including oligonucleotides, and plasmids previously established to be in vivo substrates for telomere resolution in B. burgdorferi (Chaconas et al., 2001), as well as more recently designed substrates (see FIGS. 2B and 2C). Accordingly, the present invention provides a method of identifying a modulator of ResT comprising:

(a) incubating a test substance in the presence of ResT and a telomere resolution substrate; and

(b) determining the effect of the test substance on telomere resolution.

In embodiments of the present invention, the telomere resolution substrate comprises a functional replicated telomere having at least about 38 bp of a replicated telomere from Borrelia. In further embodiments, the telomere resolution substrate may be incorporated within a plasmid or attached to a solid support.

The present invention also includes a kit for use in identifying a modulator of telomere resolvase comprising an aliquot of ResT and an aliquot of a telomere resolution substrate comprising a functional replicated telomere from Borrelia.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows In vitro telomere resolution by ResT. A) The replication strategy for linear replicons with covalently closed hairpin ends in Borrelia burgdorferi. The L and R arrows indicate the inverted repeats at the left and right ends, respectively. The line bisecting the head-to-head (L′-L) and tail-to-tail (R-R′) telomere junctions in the replication intermediate is an axis of 180% rotational symmetry. The telomere breakage and reunion reaction is referred to as telomere resolution. This figure is adapted from (Chaconas et al., 2001; Kobryn and Chaconas, 2001). B) Schematic of the telomere resolution reaction on pGCL15-6, a plasmid with a 70 bp replicated telomere which undergoes telomere resolution in vivo (Chaconas et al., 2001). Treatment of pGCL15-6 with ResT produces a linear plasmid with hairpin termini that can be removed by digestion with XbaI. Digestion of the ResT reaction product with PstI produces fragments of 2.6 kb and 2.0 kb. C) Panels from an ethidium bromide stained 1% agarose gel are presented. The left panel shows ResT reactions with topoisomerase I relaxed parent (pKK81) or substrate (pGCL15-6) plasmids. PstI digestion of the ResT treated pGCL15-6 is shown in lane 5. The central panel shows the thermal snap-back properties of the ResT reaction product (lane 9) and the effect upon this rapid renaturation of removal of the hairpin termini by XbaI digestion (lane 7). Heat treatment was at 95% C for 10 min in the presence of 15% formamide, followed by rapid cooling to 0% C prior to gel loading. The right panel shows ResT treatment of the supercoiled form of pGCL15-6. R, L and S indicate relaxed, linear and supercoiled plasmid, respectively, and ss denotes single stranded DNA. The numbers 2.6 and 2.0 on panel 2 indicate the size in kilobasepairs of the bands in lane 5.

FIG. 2A is the amino acid sequence of ResT (SEQ ID NO: 1).

FIG. 2B shows the nucleic acid sequences of the replicated telomere resolution pGCL15-6, 70 bp (SEQ ID NO: 2), pYT1, 50 bp (SEQ ID NO: 3) and pYT10, 38 bp (SEQ ID NO: 4) substrates, as well as their corresponding unreplicated forms (35 bp (SEQ ID NO: 5), 25 bp (SEQ ID NO: 6) and 19 bp (SEQ ID NO: 7)), derived from the sequence at the left end of Ip17 (Hinnebusch and Barbour, 1991).

FIG. 2C shows examples of functional and non-functional telomere derivatives for use as substrates for ResT. The sequences are also shown in SEQ ID NOS: 6-18. The derivatives are shown in their unreplicated form.

FIG. 3 shows the sequence comparison of the putative hairpin binding domains of Tn5, Tn10 and ResT (SEQ ID NOS: 19-21).

FIG. 4 is an SDS-PAGE showing the purity of recombinant ResT.

FIG. 5 shows the native and denaturing agarose gel analysis of the ResT reaction product. A) Schematic of the telomere resolution reaction on SspI linearized pGCL47-4, a plasmid with a 70 bp replicated telomere. Treatment of SspI linearized pGCL47-4 with ResT produces two double stranded DNA fragments of 0.8 and 1.9 kb, each with a hairpin telomere at one end. Denaturation of these products yields single stranded DNA species with a chain length of 1.6 and 3.8 kb, twice that of the double stranded molecules. B) Reverse images of ethidium bromide stained 1% native and alkaline agarose gels. Reactions in the absence (−) and presence (+) of ResT using 1 ug of SspI linearized pGCL47-4 were performed and split between the native and alkaline gels. 250 ng per lane were loaded on the native gel and 500 ng per lane on the alkaline gel. The alkaline gel was stained in ethidium bromide after neutralization (45 min in 1 M Tris-HCl [pH 7.6], 1.5M NaCl). The size of the molecular weight markers is noted to the left of each gel.

FIG. 6 illustrates a demonstration of a covalent ResT-DNA intermediate in telomere resolution. A) Sequence comparison of ResT reveals boxes A & C (SEQ ID NOS: 22 and 23) (Esposito and Scocca, 1997; Nunes-Duby et al., 1998) corresponding to the active site of tyrosine recombinases, typified by lambda integrase. Small asterisks denote residues corresponding to the first R and the last H of the RHRH tetrad and the large asterisk indicates the putative tyrosine nucleophile at position 335 (Gopaul and Duyne, 1999; Grainge and Jayaram, 1999). Residues boxed in black are identical in >50% of the tyrosine recombinases and residues boxed in grey are similar in >50% of the sequences (Esposito and Scocca, 1997). B) A covalent protein-DNA complex is shown on this 12% SDS-PAGE gel. ResT reactions were performed with symmetrically 3′ end labeled NcoI-XbaI fragment of pGCL47-4 carrying a 70 bp replicated telomere (see supplemental Experimental procedures). All reactions were terminated after 30 sec by addition of 1% SDS followed by precipitation of the SDS with KCl to enrich for covalent protein-DNA complexes. Half of the wild type ResT reaction was treated with 2 PUK of pronase at 37% C for 20 min after enrichment (lane 3). M denotes a DNA sizing ladder; P-D, protein -DNA complex; S, substrate; DSB, double strand break products. C) The polarity of the protein attachment was analyzed using symmetrically 5′ end labeled NcoI digested pGCL47-4 carrying a 70 bp replicated telomere. ResT reactions were terminated by addition of SDS to 0.5%. Lane 3 was treated with pronase as noted for Panel B. Products were analyzed on a 7.5% sequencing gel. HP denotes hairpin product, CL the cleaved intermediate and M an A>C sequencing ladder of the hairpin product.

FIG. 7 shows the mechanism of action of ResT. A) Mapping the ResT induced nick site in the 70 bp left end replicated telomere from B. burgdorferi Ip17. An asymmetrically 3′ end labeled NcoI-SspI fragment from pGCL47-4, which carries the 70 bp replicated telomere, was reacted with ResT at 30% C for 30 seconds as noted in FIG. 3. Products were analyzed on a 7.5% sequencing gel along with a nucleotide ladder (N) displaying prominent T residues and an A>C Maxam-Gilbert ladder of the hairpin product (see Example 9). HP denotes hairpin product and CL the cleaved intermediate. The circled A nucleotide on the sequence (position indicated by an asterisk on the gel) indicates that the sugar ring of this nucleotide is broken in the A>C reaction. This leaves a phosphate group on the 5′ end of the resultant DNA chain. The ResT cleavage intermediate terminates with a hydroxyl group instead; it therefore shows a slightly slower migration between successive bands in the marker lane due to the absence of the additional negative charge. B) The arrows indicate the position of ResT induced DNA cleavage on the replicated-telomere (the central 32 bp of the 70 bp replicated telomere present in pGCL47-4 are shown). The hatched line indicates the axis of 180% rotational symmetry for the inverted repeat. C) Proposed mechanism of telomere resolution by ResT. In a relaxed or linearized plasmid the telomere junction is presented as lineform DNA with a head-to-head structure for the inverted repeat (noted by the thin arrows). The scissile phosphates are noted with black dots, and are 6 nucleotides apart on opposite strands, placing them on the same face of the DNA double helix. The shaded ovals represent ResT protomers and the unshaded portions denote the active site with its putative tyrosine nucleophile (circled Y). The open arrows indicate the orientation of the ResT protomers. For simplicity, the reaction is drawn with active site function in cis. DNA cleavage is effected through nucleophilic attack by an active site tyrosine residue which makes a covalent intermediate with the DNA through a 3′ phosphotyrosine linkage. The 5′ hydroxyl groups are brought into proximity with the phosphotyrosine linkage for transesterification by a conformational change in the complex or by simple dissociation, with joining of the bottom strand to the top strand to produce the DNA hairpin. D) In a supercoiled plasmid the telomere junction is presented as cruciform DNA with the inverted repeats in the opposite orientation to that found in the lineform DNA. This structure would block interaction of ResT protomers by reversing their relative orientation. They would also be separated in space on the long arms of the extruded cruciform. Additionally, the cleavage sites are also moved far from the strand they need to be joined to for hairpin formation.

FIG. 8A is an agarose gel showing inhibition of ResT activity by coumermycin A1 and novobiocin.

FIG. 8B is a graph showing inhibition of growth of B. burgdorferi versus concentration of coumermycin and novobiocin.

FIG. 9 is a table showing inhibition of ResT by various synthetic peptides (SEQ ID NOS: 24-31).

### DETAILED DESCRIPTION OF THE INVENTION

I. ResT Protein

The present inventors were the first to positively identify the gene which encodes the protein responsible for telomere resolution in Borrelia burgdorferi by unequivocally showing that the BBB03 locus, carried by the circular plasmid cp26, encodes the B. burgdorferi telomere resolvase. The present inventors were also the first to produce the recombinant B. burgdorferi telomere resolvase, referred to herein as ResT. The present invention therefore provides an isolated ResT protein involved in telomere resolution in Borrelia burgdorferi. The invention covers all uses of this protein as a telomere resolvase as well all uses of various structural forms of ResT which retain biological activity.

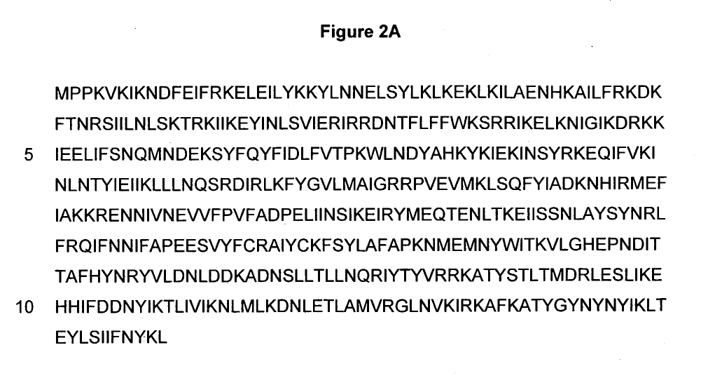

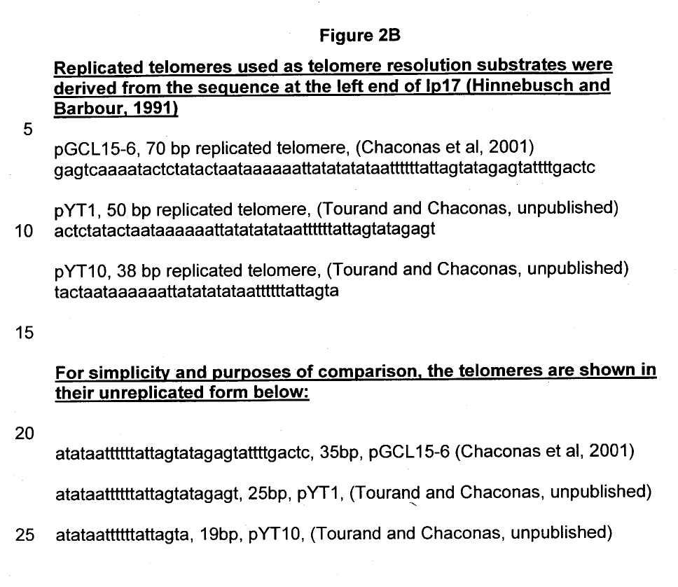

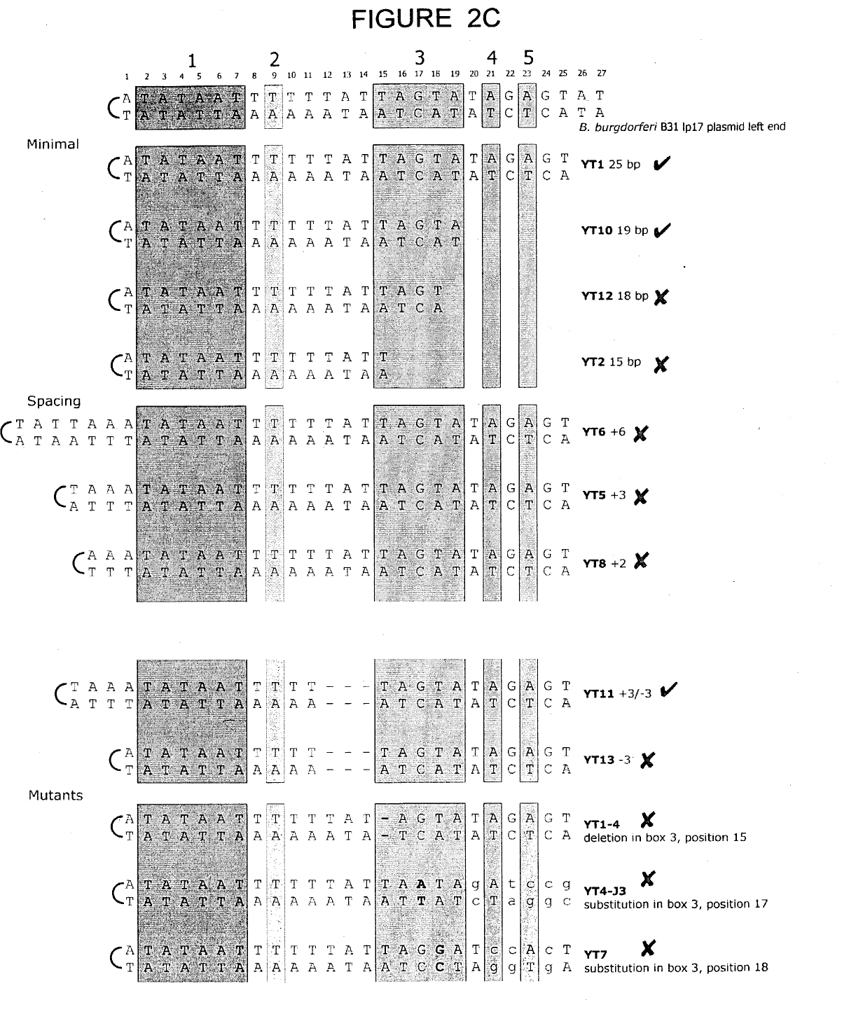

The term “ResT” as used herein means the telomere resolvase isolated from B. burgdorferi having the amino acid sequence shown in FIG. 2A (SEQ ID NO: 1) as well as any analog, homolog, isoform or fragment of the protein shown in SEQ ID NO: 1 that retains the telomere resolvase function.

Accordingly, the present invention provides an isolated ResT protein having the amino acid sequence shown in FIG. 2A (SEQ ID NO: 1) or an analog, homolog, isoform or truncation thereof.

Analogs of the protein having the amino acid sequence shown in SEQ. ID. NO: 1 and/or truncations thereof, may include, but are not limited to an amino acid sequence containing one or more amino acid substitutions, insertions, and/or deletions. Amino acid substitutions may be of a conserved or non-conserved nature. Conserved amino acid substitutions involve replacing one or more amino acids of the protein of the invention with amino acids of similar charge, size, and/or hydrophobicity characteristics. When only conserved substitutions are made the resulting analog should be functionally equivalent. Non-conserved substitutions involve replacing one or more amino acids of the amino acid sequence with one or more amino acids which possess dissimilar charge, size, and/or hydrophobicity characteristics.

One or more amino acid insertions may be introduced into the amino acid sequence shown in SEQ. ID. NO: 1. Amino acid insertions may consist of single amino acid residues or sequential amino acids ranging from 2 to 15 amino acids in length.

Deletions may consist of the removal of one or more amino acids, or discrete portions from the amino acid sequence shown in SEQ. ID. NO: 1. The deleted amino acids may or may not be contiguous. The lower limit length of the resulting analog with a deletion mutation is about 15 amino acids, preferably 50 amino acids.

Analogs of a protein of the invention may be prepared by introducing mutations in the nucleotide sequence encoding the protein. Mutations in nucleotide sequences constructed for expression of analogs of a protein of the invention must preserve the reading frame of the coding sequences. Furthermore, the mutations will preferably not create complementary regions that could hybridize to produce secondary mRNA structures, such as loops or hairpins, which could adversely affect translation.

Mutations may be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion.

Alternatively, oligonucleotide-directed site specific mutagenesis procedures may be employed to provide an altered gene having particular codons altered according to the substitution, deletion, or insertion required. Deletion or truncation of a protein of the invention may also be constructed by utilizing convenient restriction endonuclease sites adjacent to the desired deletion. Subsequent to restriction, overhangs may be filled in, and the DNA religated. Exemplary methods of making the alterations set forth above are disclosed by Sambrook et al (Molecular Cloning: A Laboratory Manual, 2nd Ed., Cold Spring Harbor Laboratory Press, 1989).

Specific mutations could be introduced, for example, that will increase the yield of production of the recombinant protein and/or improve the activity of the protein as a telomere resolvase.

The proteins of the invention also include homologs of the amino acid sequence shown in SEQ. ID. NO: 1 and/or truncations thereof. A homologous protein includes a protein with an amino acid sequence having at least 70%, preferably 80-90% identity with the amino acid sequence as shown in SEQ. ID. NO: 1.

The invention also contemplates isoforms of the protein of the invention. An isoform contains the same number and kinds of amino acids as a protein of the invention, but the isoform has a different molecular structure. The isoforms contemplated by the present invention are those having the same properties as a protein of the invention as described herein.

Truncated ResT proteins may comprise peptides of at least 15 amino acid residues of the proteins of the invention.

The present invention also includes a protein of the invention conjugated with a selected protein, or a selectable marker protein (see below) to produce fusion proteins. Additionally, immunogenic portions of a protein of the invention are within the scope of the invention.

The proteins of the invention (including truncations, analogs, etc.) may be prepared using recombinant DNA methods, for example as described in Example 2 hereinbelow. Accordingly, nucleic acid molecules encoding the proteins of the present invention may be incorporated in a known manner into an appropriate expression vector which ensures good expression of the protein in a host cell.

II. Screening Assay

As previously mentioned, the present inventors have purified the B. burgdorferi ResT protein and are the first to show that it is a highly efficient telomere resolvase in a reaction that does not require accessory proteins, divalent metal ions or a high energy cofactor. The activity of the purified recombinant protein can be assayed using plasmids previously established to be in vivo substrates for telomere resolution in B. burgdorferi (Chaconas et al, 2001).

The in vitro reaction mimics telomere resolution in vivo (Chaconas et al., 2001) and is a conservative, sequence-specific DNA breakage and reunion reaction that generates two hairpin telomeres from a replicated telomere substrate. The ResT protein represents a new class of DNA breakage and reunion enzymes, which currently consists of B. burgdorferi ResT and the E. coli phage N15 TeIN (Deneke et al., 2000). While ResT seems to use a similar reaction chemistry to topoisomerases and site-specific recombinases, it performs a unique reaction. Topoisomerases promote breakage and reunion of either one or two DNA strands to alter the topological state of a DNA molecule. Site-specific recombinases perform a more complex reaction in which four strands are broken and subsequently joined to a different DNA duplex, resulting in the production of a recombinant product. The telomere resolvases TeIN and ResT, on the other hand, must break two phosphodiester bonds in a single DNA duplex (one on each strand) and join each end with the opposite DNA strand to form covalently closed hairpin telomeres.

Accordingly, the present invention provides a method of identifying a modulator of ResT comprising:

(a) incubating a test substance in the presence of ResT and a telomere resolution substrate; and

(b) determining the effect of the test substance on telomere resolution, wherein a change in telomere resolution as compared to a control means the test substance is a modulator of ResT activity.

The phrase “determining the effect of the test substance on telomere resolution” means that the effect of the test substance on the activity of ResT will be assayed and compared to the activity that is normally observed in the absence of the test substance. Preferably the screening assay is repeated using a control sample with the same conditions and components as the test sample but without the test substance. The activity of ResT in the presence of the test substance is then directly compared to the control.

The term “activity of ResT” or “ResT activity” means the telomere resolvase activity of ResT.

The term “modulator of ResT” as used herein means any substance that can modulate the activity of the telomere resolvase enzyme, ResT. The term includes both substances that can activate or enhance the activity of ResT as well as substances that can inhibit or suppress the activity of ResT. Such modulators include, but are not limited to, proteins (including antibodies), peptides, nucleic acids (including RNA, DNA, genes, oligonucleotides, antisense oligonucleotides, peptide nucleic acids), carbohydrates, organic compounds, inorganic compounds and natural products.

In a preferred embodiment, the assay of the invention is used to identify inhibitors of ResT. The term “inhibitor of ResT” or “ResT inhibitor” means any substance or agent that causes a decrease in, or inhibition of, ResT activity as compared to the activity in the absence of the substance or agent.

The ResT enzyme used in the assay can be in purified or isolated form such as recombinant ResT. Alternatively, Borrelia cells that produce ResT can be used as the ResT source for the assay.

In embodiments of the present invention, the telomere resolution substrate comprises a functional replicated telomere. The functional replicated telomere is preferably from a Borrelia species. In specific embodiments, the substrate comprises at least about 38 bp, preferably about 50 bp, of a replicated telomere, preferably the left telomere, from the linear plasmid Ip17 of B. burgdorferi (see FIG. 2B and SEQ ID NO: 2). ResT is sequence specific, therefore the telomere resolution substrate must comprise at least a functional portion of a replicated telomere from Borrelia. The nucleic acid sequences of replicated telomeres derived from the sequence at the left end of linear plasmid Ip17 of B. burgdorferi used as telomere resolution substrates herein are shown in FIG. 2B; pGCL15-6 (SEQ ID NO. 2); pYT1 (SEQ ID NO: 3); and pYT10 (SEQ ID NO: 4) in replicated forms and, pGCL15-6 (SEQ ID NO: 5); pYT1 (SEQ ID NO: 6); and pYT10 (SEQ ID NO: 7) in unreplicated forms. The substrate may also include functional derivatives of a replicated telomere from Borrelia, including derivatives wherein nucleotides have been inserted into and/or deleted from a replicated telomere from Borrelia. For example, insertion and deletion analogs of the sequences shown in FIG. 2B. Examples of some functional and non-functional telomere derivatives (shown before replication) are found in FIG. 2C. Telomeres with two distinct types of DNA spacing, for example, pYT1 (SEQ ID NO: 3) and pYT11 (SEQ ID NO: 14), are used by ResT in vitro, and correspond to the two types of spacing found in naturally occurring telomeres (see Casjens, 1999). A person having skill in the art would be able to identify functional derivatives of a replicated telomere by preparing the derivative and assaying its use as a substrate for ResT, for example as described in Example 4, herein.

In further embodiments of the present invention, the telomere resolution substrate may also comprise a label, for example a fluorescent or radioactive label, which can be used to monitor the progress of the telomere resolution reaction.

In still further embodiments of the present invention, the telomere resolution substrate may be incorporated within a plasmid. When the substrate is incorporated within a plasmid, the plasmid may be in a form selected from circular, open circular and linearized forms.

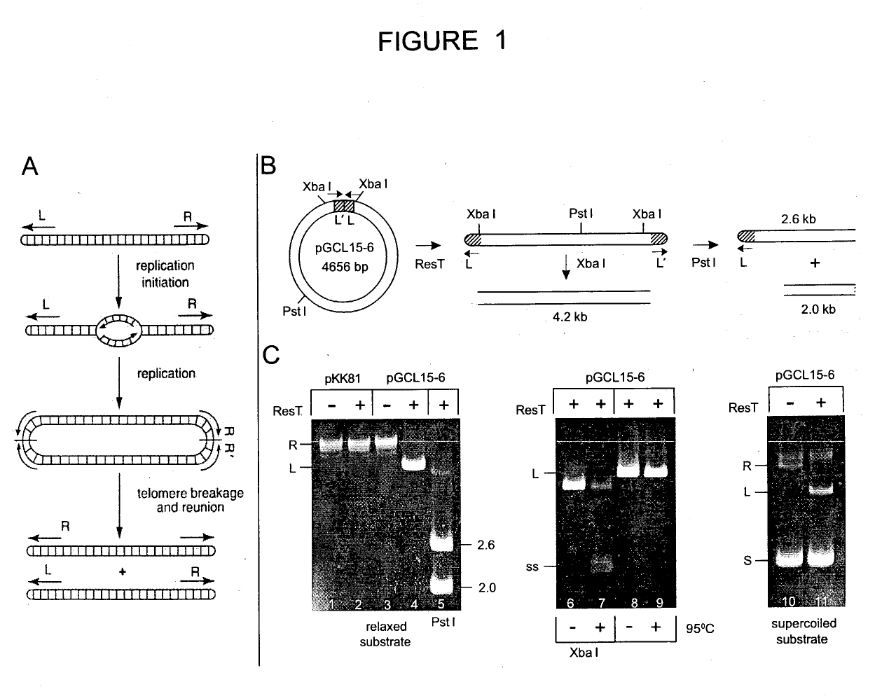

The activity of ResT can be assayed by monitoring the appearance of the expected DNA product from the action of ResT on the replicated telomere substrate. Any known method for detecting nucleic acid molecules may be used to monitor the appearance of the expected product. For example, fluorescent substrates or radioactive substrates may be used and the products assayed using standard methods. Further, assay methods utilizing the snap-back properties of the hairpin products of ResT (demonstrated in FIG. 1C) are also included in the present invention. Other forms of electrophoresis (eg capillary electrophoresis) or other technologies, such as the use of a mass spectrometer, a fluorescence reader or other methods to monitor ResT activity are similarly included within the scope of the present application.

Accordingly, in an embodiment of the present invention, there is provided a method of identifying a modulator of ResT comprising:

(a) incubating a test substance in the presence of ResT and a telomere resolution substrate comprising a functional replicated telomere; and

(b) assaying for the presence of an expected product;

wherein a change in an amount of expected product in the presence of the test substance compared to a control indicates that the test substance is a modulator of ResT. By “control” it is meant performing the method using the same conditions and components as with the test substance, but without the test substance.

When the substrate is a circular plasmid comprising at least about 38 bp of a replicated left end telomere from the linear plasmid Ip17 of B. burgdorferi, the reaction can be monitored by assaying for the presence of the expected linear DNA molecule comprising two hairpin telomeres. Alternatively, the linear product may be treated with a restriction enzyme and the presence of the expected DNA fragments may be assayed. For example, the linearized product obtained from the reaction of ResT with a circular plasmid comprising a 70 bp replicated left end telomere from the linear plasmid Ip17 of B. burgdorferi (see FIG. 2B) may be treated with PstI and the reaction mixture assayed for the presence of the expected 2.0 and 2.6 kb DNA fragments. The presence of the expected DNA products from the ResT reactions can be assayed using any known technique, for example, using ethidium bromide stained native or alkaline agarose gels, PCR and/or DNA sequencing.

In embodiments of the present invention, the telomere resolution substrate is a circular plasmid and the expected product is a linearized plasmid. Preferably the circular plasmid comprises at least about a 38 bp replicated telomere from the linear plasmid Ip17 of B. burgdorferi and the expected product is a linear DNA molecule comprising two telomeres.

In a further embodiment of the present invention, the expected product from the reaction of ResT with a telomere resolution substrate comprising a functional replicated telomere in the presence of a test substance is further treated with a restriction enzyme to provide one or more DNA fragments having known sizes and a change in an amount of one or more DNA fragments in the presence of the test substance compared to a control indicates that the test substance is a modulator of Borrelia telomere resolvase.

When the telomere resolution substrate is a circular plasmid comprising a 70 bp replicated left end telomere from the linear plasmid Ip17 of B. burgdorferi and the restriction enzyme is PstI, the resulting DNA fragments are 2.0 and 2.6 kb in size.

In further embodiments of the present invention, the telomere resolution substrate is attached to a solid support. Attaching the telomere resolution substrate to a solid support is especially useful in high-throughput screening for modulators of ResT, for example, an oligonucleotide substrate of at least about 38 base pairs may be bound to the wells (384 or greater) of streptavidin coated assay plates. The oligonucleotide substrate may contain a label, for example a biotin label, at one end to tether the substrate to the streptavidin coated wells of the assay plate and another label, for example a fluorescent or radioactive label, at the other end for detection purposes. The action of ResT on this substrate will sever the fluorescent or radioactive label from the portion of the substrate which is linked to the plate. The fluorescent or radioactive label would then be removable from the well by a simple washing step. Determination of fluorescence using a fluorescence micro-plate reader or radioactivity using standard counters, allows a facile assay for ResT activity. When the enzyme is active, the fluorescence or radioactivity will decrease after incubation with ResT. If the reaction is blocked by an inhibitor the fluorescence or radioactivity level will remain constant or show substantially less reduction than in the absence of inhibitor (control).

It has been found that spermidine contributes an approximate 3-fold stimulation to the in vitro telomere resolution reaction involving ResT. Therefore, in further embodiments of the present invention, the test substance is incubated in the presence of ResT, a telomere resolution substrate and spermidine.

The test substance can be any compound which one wishes to test including, but not limited to, proteins (including antibodies), peptides, nucleic acids (including RNA, DNA, antisense oligonucleotide, peptide nucleic acids), carbohydrates, organic compounds, inorganic compounds, natural products, library extracts, bodily fluids and other samples that one wishes to test for modulators of ResT. More than one test compound can be tested at a time in the assay of the invention. As such the assay is useful in testing the combined effects of two or more compounds on the modulation of Borrelia telomere resolvase.

As previously mentioned, the method is adaptable to high-throughput screening applications. For example, a high-throughput screening assay may be used which comprises any of the methods according to the invention wherein aliquots of ResT and telomere resolution substrate are exposed to a plurality of test compounds within different wells of a multi-well plate. Further, a high-throughput screening assay according to the invention involves aliquots of ResT and telomere resolution substrate which are exposed to a plurality of candidate substances in a miniaturized assay system of any kind. Another embodiment of a high-throughput screening assay could involve exposing aliqouts of ResT and telomere resolution substrate simultaneously to a plurality of test compounds.

The method of the invention may be “miniaturized” in an assay system through any acceptable method of miniaturization, including but not limited to multi-well plates, such as 24, 48, 96 or 384-wells per plate, micro-chips or slides. The assay may be reduced in size to be conducted on a micro-chip support, advantageously involving smaller amounts of reagents and other materials. Any miniaturization of the process which is conducive to high-throughput screening is within the scope of the invention.

ResT is likely to be the only site-specific telomere resolvase encoded by B. burgdorferi and other Borrelia species. There are currently two arguments for this: the first is that all Borrelia telomeres sequenced to date have extensive homology in the first two dozen base pairs (Casjens, 1999; Casjens et al., 1997), suggesting that they are all recognized by the same protein. The second is that searches of the B. burgdorferi genome do not reveal any proteins with significant homology to ResT. Therefore a modulator of ResT from Borrelia burgdorferi would be an effective modulator of telomere resolvases in all species of Borrelia and may also be an effective modulator of telomere resolvases in all species for which a telomere resolvase is involved in replication of linear replicons.

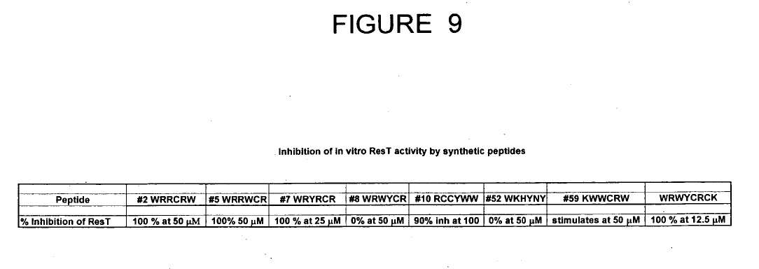

In a specific embodiment, the screening assay is used to identify inhibitors of ResT. As described in Examples 10 and 11, using the screening assay of the invention, the inventors determined that two coumarin antibiotics, coumermycin Al and novobiocin and several peptides (see FIG. 9 and SEQ ID NOS: 24-31) inhibited ResT activity. The inventors further demonstrated that coumermycin and novobiocin could inhibit the growth of B. burgdorferi. Therefore, these results demonstrate that the screening assay of the invention is useful in identifying ResT inhibitors that are useful in identifying potential therapeutic or environmental agents for treating Borrelia infections.

In an alternate embodiment, ResT inhibitors may be identified by adding the test substance to a culture of Borrelia cells, harvesting the culture after an appropriate period of time (a few hours) and assaying for circular dimeric DNA replication intermediates which would accumulate if the test substance inhibits ResT.

The development of the screening assay of the invention allows the preparation of kits for use in identifying modulators of the activity of ResT. The kits would comprise the reagents suitable for carrying out the methods of the invention, packaged into suitable containers and providing the necessary instructions for use.

Accordingly, the present invention includes a kit for use in identifying a modulator of ResT comprising an aliquot of ResT and an aliquot of a telomere resolution substrate preferably comprising a functional replicated telomere, from Borrelia.

In embodiments of the present invention, the substrate comprises at least about 38 bp, preferably at least about 50 bp, of a replicated telomere, preferably the left end telomere, from the linear plasmid Ip17 of B. burgdorferi. The substrate may be, for example, incorporated within a plasmid (for example the plasmid pGCL15-6 used in Example 4) or attached to a solid support and may further comprise a label for monitoring the progress of the reaction. The kit may provide instructions for carrying out the assay of the invention. The kit may optionally include spermidine, and other reagents such as buffers and the like for performing the assay of the invention.

With particular regard to assay systems packaged in “kit” form, it is preferred that assay components be packaged in separate containers, with each container including a sufficient quantity of reagent for at least one assay to be conducted. A preferred kit is typically provided as an enclosure (package) comprising one or more containers for the within-described reagents.

The reagents as described herein may be provided in solution, as a liquid dispersion or as a substantially dry powder, e.g., in lyophilized form. Usually, the reagents are packaged under an inert atmosphere.

Printed instructions providing guidance in the use of the packaged reagent(s) may also be included, in various preferred embodiments. The term “instructions” or “instructions for use” typically includes a tangible expression describing the reagent concentration or at least one assay method parameter, such as the relative amounts of reagent and sample to be admixed, maintenance time periods for reagent/sample admixtures, temperature, buffer conditions, and the like.

III. Modulators of ResT

In addition to the ResT modulators that can be identified by the above described screening assays of the invention, other ResT modulators can be prepared based on the sequence or structure of ResT. The preparation of some additional ResT modulators are described below. Once prepared, these modulators can be tested for their ability to modulate ResT activity using the screening assays described above.

(a) Antibodies

The present invention includes an antibody that binds to ResT as a potential ResT modulator. Antibodies to the various ResT may be useful therapeutically as discussed in greater detail in Section VI.

Antibodies to ResT can be prepared using techniques known in the art. For example, by using a peptide of ResT polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the protein or peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for ResT as described herein.

The term “antibody” as used herein is intended to include fragments thereof which also specifically bind with ResT or peptide thereof. Antibodies can be fragmented using conventional techniques and the fragments screened for utility in the same manner as described above. For example, F(ab′)² fragments can be generated by treating antibody with pepsin. The resulting F(ab′)₂ fragment can be treated to reduce disulfide bridges to produce Fab′ fragments.

Chimeric antibody derivatives, i.e., antibody molecules that combine a non-human animal variable region and a human constant region are also contemplated within the scope of the invention. Chimeric antibody molecules can include, for example, the antigen binding domain from an antibody of a mouse, rat, or other species, with human constant regions. Conventional methods may be used to make chimeric antibodies containing the immunoglobulin variable region which recognizes the gene product of ResT antigens of the invention (See, for example, Morrison et al., Proc. Natl Acad. Sci. U.S.A. 81,6851 (1985); Takeda et al., Nature 314, 452 (1985), Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). It is expected that chimeric antibodies would be less immunogenic in a human subject than the corresponding non-chimeric antibody.

Monoclonal or chimeric antibodies specifically reactive with ResT as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art, (e.g., Teng et al., Proc. Natl. Acad. Sci. U.S.A., 80, 7308-7312 (1983); Kozbor et al., Immunology Today, 4, 7279 (1983); Olsson et al., Meth. Enzymol., 92, 3-16 (1982)), and PCT Publication WO92/06193 or EP 0239400). Humanized antibodies can also be commercially produced (Scotgen Limited, 2 Holly Road, Twickenham, Middlesex, Great Britain.)

Specific antibodies, or antibody fragments, reactive against ResT proteins may also be generated by screening expression libraries encoding immunoglobulin genes, or portions thereof, expressed in bacteria with peptides produced from the nucleic acid molecules of ResT. For example, complete Fab fragments, VH regions and FV regions can be expressed in bacteria using phage expression libraries (See for example Ward et al., Nature 341, 544-546: (1989); Huse et al., Science 246, 1275-1281 (1989); and McCafferty et al. Nature 348, 552-554 (1990)). Alternatively, a SCID-hu mouse, for example the model developed by Genpharm, can be used to produce antibodies or fragments thereof.

(b) Antisense Oligonucleotides

The invention also includes antisense oligonucleotides that can modulate the expression and/or activity of ResT. Accordingly, the present invention provides an antisense oligonucleotide that is complimentary to a nucleic acid sequence encoding ResT or an antisense oligonucleotide that is complimentary to a ResT substrate.

The term “antisense oligonucleotide” as used herein means a nucleotide sequence that is complimentary to its target.

The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells), or two or more oligonucleotides of the invention may be joined to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates, and phosphorodithioates. In an embodiment of the invention there are phosphorothioate bonds links between the four to six 3′-terminus bases. In another embodiment phosphorothioate bonds link all the nucleotides.

The antisense oligonucleotides of the invention may also comprise nucleotide analogs that may be better suited as therapeutic or experimental reagents. An example of an oligonucleotide analogue is a peptide nucleic acid (PNA) wherein the deoxyribose (or ribose) phosphate backbone in the DNA (or RNA), is replaced with a polyamide backbone which is similar to that found in peptides (P. E. Nielsen, et al Science 1991, 254, 1497). PNA analogues have been shown to be resistant to degradation by enzymes and to have extended lives in vivo and in vitro. PNAs also bind stronger to a complimentary DNA sequence due to the lack of charge repulsion between the PNA strand and the DNA strand. Other oligonucleotides may contain nucleotides containing polymer backbones, cyclic backbones, or acyclic backbones. For example, the nucleotides may have morpholino backbone structures (U.S. Pat. No. 5,034,506). Oligonucleotides may also contain groups such as reporter groups, a group for improving the pharmacokinetic properties of an oligonucleotide, or a group for improving the pharmacodynamic properties of an antisense oligonucleotide. Antisense oligonucleotides may also have sugar mimetics.

The antisense nucleic acid molecules may be constructed using chemical synthesis and enzymatic ligation reactions using procedures known in the art. The antisense nucleic acid molecules of the invention or a fragment thereof, may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The antisense oligonucleotides may be introduced into tissues or cells using techniques in the art including vectors (retroviral vectors, adenoviral vectors and DNA virus vectors) or physical techniques such as microinjection. The antisense oligonucleotides may be directly administered in vivo or may be used to transfect cells in vitro which are then administered in vivo. In one embodiment, the antisense oligonucleotide may be delivered to macrophages and/or endothelial cells in a liposome formulation.

(c) Peptide Mimetics

The present invention also includes peptide mimetics of the ResT proteins of the invention. Such peptides may include competitive inhibitors, enhancers, peptide mimetics, and the like. All of these peptides as well as molecules substantially homologous, complementary or otherwise functionally or structurally equivalent to these peptides may be used for purposes of the present invention.

“Peptide mimetics” are structures which serve as substitutes for peptides in interactions between molecules (See Morgan et al (1989), Ann. Reports Med. Chem. 24:243-252 for a review). Peptide mimetics include. synthetic structures which may or may not contain amino acids and/or peptide bonds but retain the structural and functional features of a peptide, or enhancer or inhibitor of the invention. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a peptide of the invention.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

(d) Drug Design

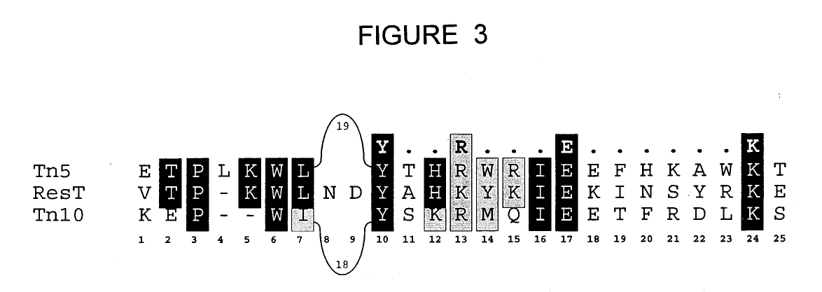

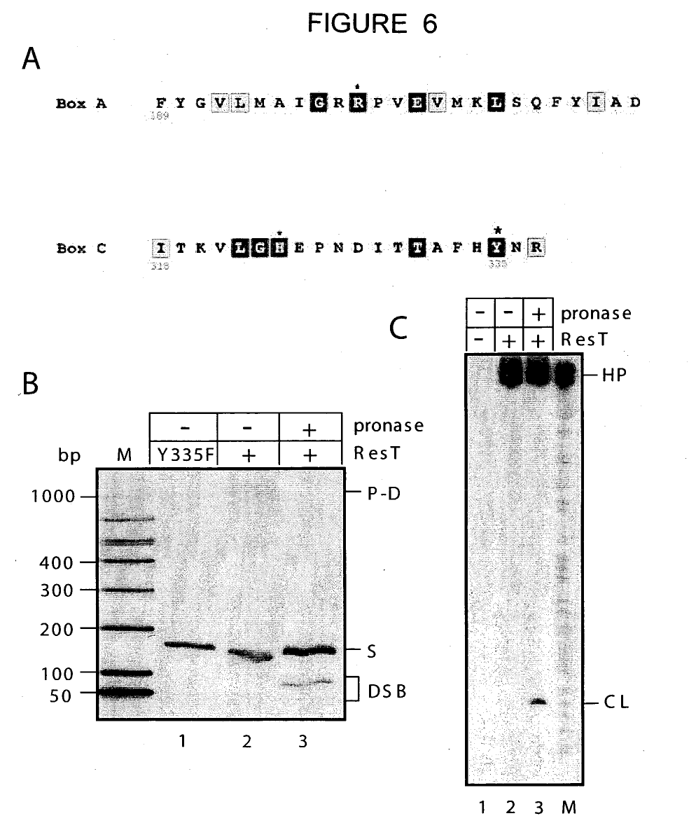

Peptides derived from the ResT isoforms may also be used to identify lead compounds for drug development. Sequence analysis reveals that ResT contains a hairpin binding domain similar to that found in the Tn5 (Davies et al., 2000) and Tn10 (Allingham et al., 2001) transposase. The conserved Y-(2)-R-(3)-E-(6)-K signature found in the transposases of IS4 family members Rezsohazy et al., 1993) is indicated in bold above the alignment shown in FIG. 3. Position 1 in the alignment corresponds to residue 137 for ResT, residue 293 for Tn5 transposase and residue 262 for Tn10 transposase. This sequence comparison forms the basis for the identification of the first hairpin binding domain outside a transposase. This hairpin binding domain may be used as a target for rationale drug design aimed at obtaining modulators of ResT activity using the assay described herein. The portion of the ResT active site resembling that of the tyrosine recombinases (Box A and C, FIG. 6A) may also be used as a target for rationale drug design aimed at obtaining modulators of ResT activity using the assay described herein.

The structure of ResT can be further elucidated by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule. The activity of the lead compounds can be evaluated using the screening assays described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess the ability to modulate ResT and/or have utility in the therapeutic applications described below.

IV. Uses

The present invention includes all uses of the ResT protein, the screening assay, the kit and ResT modulators of the invention, some of which are described below.

(a) Therapeutic Uses

An interesting feature of the ResT telomere resolvase is that it is encoded by the B. burgdorferi circular plasmid cp26 (Casjens et al., 2000) rather than by the linear chromosome. The resT gene (BBB03) maps between BBB02, of unknown function, and chbC (BBB04), encoding a component of the chitobiose transport system (Tilly et al., 2001). Other important genes such as ospC (Sadziene et al., 1993), and those involved in purine biosynthesis (Margolis et al., 1994) are also carried on cp26. Although many B. burgdorferi plasmids can be lost during in vitro cultivation, cp26 has never been reported missing in laboratory grown strains (McDowell et al., 2001; Purser and Norris, 2000). The identification of resT as the telomere resolvase gene suggests that cp26 may be an essential replicon for B. burgdorferi and that it is indeed a mini-chromosome (Barbour and Fish, 1993) rather than an expendable plasmid. The identification of modulators of ResT is therefore expected to provide substances capable of modulating of the replication of Borrelia. Of particular interest are substances that inhibit Borrelia replication.

The assay and kit of the invention allow the identification of modulators of the activity of ResT. Substances that inhibit the activity of ResT may be used, for example, in developing drugs for treating or preventing diseases and conditions caused by Borrelia infection. Such diseases and conditions include, but are not limited to Lyme disease and tick-bourne or louse-bourne relapsing fever.

Since ResT is responsible for telomere resolution, modulators of the telomere resolution reaction promoted by ResT may modulate telomere resolution in all organisms for which a telomere resolvase is involved in replication of linear replicons. Accordingly, the present invention provides a method of modulating telomere resolution comprising administering an effective amount of a modulator of ResT to a cell or animal in need thereof. The present invention also provides a method of modulating DNA replication comprising administering an effective amount of a modulator of ResT to a cell or animal in need thereof.

Because telomere resolution by ResT has been confirmed in the genus Borrelia, inhibitors of the telomere resolution reaction promoted by ResT will block DNA replication of all linear replicons in Borrelia species and hence act as highly effective anti-borrelial agents. The genus Borrelia contains spirochetes causing Lyme disease and relapsing fever (Barthold, 2000; Nordstrand et al., 2000; Schwan et al., 1999; Shapiro and Gerber, 2000) and is the only known bacterial genus characterized by linear replicons containing covalently closed hairpin ends (Barbour and Garon, 1987; Casjens et al., 1997; Hinnebusch and Barbour, 1991).

The potential uses of antimicrobial agents which are highly effective inhibitors for a process (telomere resolution) which is found in Borrelia species should not be underestimated. Current antibiotic therapies do nothing to halt the spread of Lyme disease or reduce the incidence of Lyme disease in endemic areas. An example of highly beneficial use for telomere resolvase inhibitors is the potential environmental use of such agents in the elimination of Borrelia species from infected arthropod vectors and vertebrate reservoirs (e.g. mice, birds and lizards) in regions where Lyme disease and relapsing fever are prominent health risks. The success of such an approach would eliminate diseases that are an increasing health risk, are difficult to diagnose, and may have long term debilitating effects if not treated promptly after infection. A further advantage of telomere resolvase inhibitors as therapeutic drugs against Borrelia species is the lack of concern regarding the development and transmission of generalized antibiotic resistance.

Accordingly, the present invention provides a method of treating or preventing Borrelia infection comprising administering an effective amount of an inhibitor of ResT to an animal in need thereof. The present invention also provides the use of an inhibitor of ResT to treat or prevent Borrelia infection and the use of an inhibitor of ResT to prepare a medicament to treat or prevent Borrelia infection.

The term “effective amount” as used herein is defined as an amount effective, at dosages and for periods of time necessary to achieve the desired result. The effective amount of a compound of the invention may vary according to factors such as the disease state, age, sex, and weight of the animal. Dosage regima may be adjusted to provide the optimum therapeutic response. For example, several divided doses may be administered daily or the dose may be proportionally reduced as indicated by the exigencies of the therapeutic situation.

The term “animal” as used herein includes all members of the animal kingdom, including humans. Preferably, the animal to be treated is a human or animals, such as ticks and lice which are disease vectors and mice, birds and lizards, which are reservoirs for Lyme disease.

The term “treating or treatment” as used herein means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treating” can also mean prolonging survival as compared to expected survival if not receiving treatment. “Treating” can also mean reducing the bacterial count in an animal infected with Borrelia or a poxvirus.

When treating non-human animals, the ResT inhibitor can be delivered directly to the animal or can be included in the feed or bedding of the animal. The ResT inhibitor can also be delivered to the environment for uptake by the animals, for example through ingestion or inhalation. The goal of such a therapy would be to reduce (preferably eliminate) the Borrelia in such animals which would reduce the spread or transmission of the bacteria to humans.

In one embodiment, the inhibitor of ResT is a peptide having the sequence WRRCRW (SEQ ID NO: 24); WRRWCR (SEQ ID NO: 25); WRYRCR (SEQ ID NO: 26); RCCYWW (SEQ ID NO: 28) or WRWYCRCK (SEQ ID NO: 31) as is shown in FIG. 9. In another embodiment, the inhibitor of ResT is a coumarin antibiotic such as coumermycin A1 or novobiocin.

Coumermycin and novobiocin were first discovered as inhibitors of bacterial DNA gyrase (a type II topoisomerase). They specifically act by binding to the gyrase B subunit. They also inhibit the poxviral topoisomerases (Vaccinia virus and Molluscum contagiosum virus), which are type 1b topoisomerases. The poxviral type 1b enzymes seem the most similar as they use the same type of covalent intermediate in the DNA breakage and reunion reaction as ResT, as well as showing a similar pattern of drug inhibition. Accordingly, ResT inhibitors may also be useful in inhibiting poxviral enzymes. As a result, inhibitors for ResT that are identified according to the invention might also inhibit the poxviral topoisomerases and might make effective drugs for treatment or prophylactic use against an infection caused by a poxvirus such as smallpox.

Accordingly, the present invention also provides a method of treating or preventing an infection caused by a poxvirus, preferably smallpox, comprising administering an effective amount of an inhibitor of ResT to an animal in need thereof. The present invention also provides the use of an inhibitor of ResT to treat or prevent a poxviral infection, preferably, smallpox infection, and the use of an inhibitor of ResT to prepare a medicament to treat or prevent poxviral infection, preferably smallpox infection. Preferably, the ResT inhibitor is a coumarin antibiotic such as coumermycin A1 or novobiocin.

The present invention also includes a method of inhibiting a topoisomerase comprising administering an effective amount of a ResT inhibitor to a cell or animal in need thereof.

The present invention also extends to the use of ResT modulators that enhance ResT activity. ResT or substances that increase the activity of ResT may be used, for example, to make hairpin DNA for commercial or therapeutic purposes. One application of this would be the preparation of hairpin DNA (currently AAV) to cause apoptosis of cancer cells which have p53 mutations (see (Vogelstein and Kinzler, 2001) and Raj et al. 2001).

(b) Pharmaceutical Compositions

The present invention includes pharmaceutical compositions containing the ResT protein and modulators of ResT as described above.

In one embodiment, the present invention provides a pharmaceutical composition comprising an effective amount of a ResT modulator in admixture with a suitable diluent or carrier.

In another embodiment, the present invention provides a pharmaceutical composition comprising an effective amount of a ResT inhibitor in admixture with a suitable diluent or carrier.

The invention further includes a method of preparing a pharmaceutical composition for use in modulating the activity of ResT or in treating a Borrelia infection comprising mixing a modulator of ResT with a suitable diluent or carrier.

Such pharmaceutical compositions can be for intralesional, intravenous, topical, rectal, parenteral, local, inhalant or subcutaneous, intradermal, intramuscular, intrathecal, transperitoneal, oral, and intracerebral use. The composition can be in liquid, solid or semisolid form, for example pills, tablets, creams, gelatin capsules, capsules, suppositories, soft gelatin capsules, gels, membranes, tubelets, solutions or suspensions. The composition is preferably injected in a saline solution either intravenously, intraperitoneally or subcutaneously.

The compositions of the invention can be used to treat animals that are disease vectors (such as ticks and lice) or animals that are resevoirs for lyme disease (such as mice, birds and lizards). In such an embodiment, the composition can be in the form of a liquid or solid feed or can be impregnated on to bedding or any other substance that may come in contact with the animals. The composition may also be in an inhalation or aerosol format. Alternatively, the composition may be delivered to ticks through feeding on drug treated animals. Another option is to deliver the compositions to the environment in areas with endemic Borrelia infection.

The pharmaceutical compositions of the invention can be intended for administration to humans or animals. Dosages to be administered depend on individual needs, on the desired effect and on the chosen route of administration.

The pharmaceutical compositions can be prepared by per se known methods for the preparation of pharmaceutically acceptable compositions which can be administered to animals, and such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985).

On this basis, the pharmaceutical compositions include, albeit not exclusively, the active compound or substance in association with one or more pharmaceutically acceptable vehicles or diluents, and contained in buffered solutions with a suitable pH and iso-osmotic with the physiological fluids.

A pharmaceutical composition of the invention may comprise nucleic acid molecules (such as antisense oligonucleotide) which may be directly introduced into cells or tissues in vivo using delivery vehicles such as retroviral vectors, adenoviral vectors and DNA virus vectors. They may also be introduced into cells in vivo using physical techniques such as microinjection and electroporation or chemical methods such as coprecipitation and incorporation of DNA into liposomes. Recombinant molecules may also be delivered in the form of an aerosol or by lavage. The nucleic acid molecules of the invention may also be applied extracellularly such as by direct injection into cells.

(c) Drug Discovery Business Methods

The present invention also includes all business applications of the screening assay of the invention including conducting a drug discovery business.

Accordingly, the present invention also provides a method of conducting a drug discovery business comprising:

(a) providing one or more assay systems for identifying a modulator of Borrelia telomere resolvase;

(b) conducting therapeutic profiling of modulators identified in step (a), or further analogs thereof, for efficacy and toxicity in animals; and

(c) formulating a pharmaceutical preparation including one or more modulators identified in step (b) as having an acceptable therapeutic profile. In certain embodiments, the subject method can also include a step of establishing a distribution system for distributing the pharmaceutical preparation for sale, and may optionally include establishing a sales group for marketing the pharmaceutical preparation.

The present invention also provides a method of conducting a target discovery business comprising:

(a) providing one or more assay systems for identifying modulators of Borrelia telomere resolvase;

(b) (optionally) conducting therapeutic profiling of modulators identified in step (a) for efficacy and toxicity in animals; and

(b) licensing, to a third party, the rights for further drug development and/or sales for modulators identified in step (a), or analogs thereof.

By assay systems, it is meant, the equipment, reagents and methods involved in conducting a screen of substances for the ability to modulate Borrelia telomere resolvase using the method of the invention.

The following non-limiting examples are illustrative of the present invention:

### EXAMPLES

### Example 1

Plasmids and Strains

Telomere resolution substrates pGCL15-6 (70 bp replicated telomere), pGCL10-2 (140 bp replicated telomere) and pGCL13-1(140 bp mock telomere) used here were all derived from B. burgdorferi Ip17 and have been previously reported (Chaconas et al., 2001). pGCL47-4 has the same 70 bp replicated telomere as pGCL15-6 but is carried on pJLB12g. Plasmids carrying the replicated telomeres were propagated in an E. coli sbc mutant strain as previously noted. The telomere inserts were sequenced after each plasmid preparation to verify the integrity of the replicated telomere.

### Example 2

Expression of ResT

Expression of ResT in E. coli was accomplished by cloning BBB03 into pET3d (Novagen). BBB03 was amplified from B. burgdorferi B31 MedImmune genomic DNA (Fraser et al., 1997) (gift of Raju Lathigra, MedImmune, Inc., Gaithersburg, Md.) using primers B53 (5′ aatacgttgagGGTCTCacatgcctccaaaagtgaagataaaa 3′) (SEQ ID NO: 32) and B52 (5′ gtgcccGGATCCctatagcttataattaaaaattattgataagta 3′) (SEQ ID NO: 33). These primers contain a BsaI (B53) and a BamHI (B52) site, noted in upper case letters, at the 5′ and 3′ ends of the gene, respectively. The BsaI site in primer B53 was engineered to produce an NcoI compatible overhang. The amplified product was digested with both enzymes and ligated with NcoI-BamHI digested pET3d to fuse the start codon of ResT with the ribosome binding site in the vector. The integrity of the construct was verified by DNA sequencing. ResT_{Y335F} was constructed using a modification (Wang and Malcolm, 1999) of the Quick Change XL site directed mutagenesis kit (Stratagene).

### Example 3

Purification of ResT

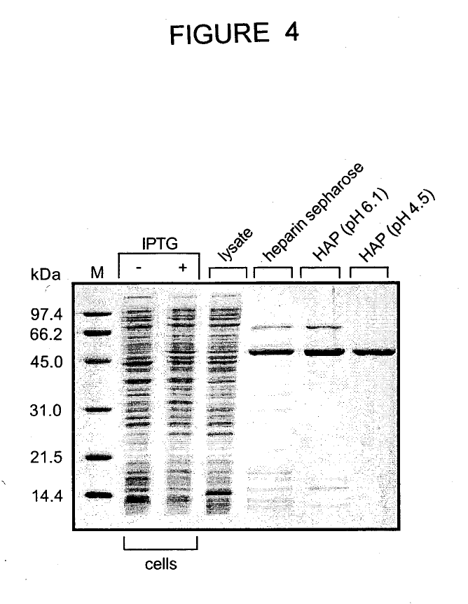

ResT was purified from BGC88, which is a λDE3 lysogen of BL21 (Novagen) carrying both pKK4, and pLysS to tightly regulate expression. A BGC88 culture (1L LB in a 2L Fernbach flask) was grown at 37% C in LB (with 100 &g/ml ampicillin, 30 &g/ml chloramphenicol and 1% glucose) to an A₅₉₅ of 0.4-0.6. The culture was then shifted to growth at 16-18% C. After one hour the culture was induced by addition of IPTG to 1 mM and incubated at 16-18% C for approximately 20 hours, until reaching an A₅₉₅ of 3.9. The lysate was prepared by multiple freeze-thaw cycles as reported previously for E. coli HU (Lavoie and Chaconas, 1993), with the addition of 1 ml of Sigma bacterial protease inhibitor cocktail (P8465) and PMSF to 0.2 mM. The lysate was adjusted to 10% w/v glycerol and 0.5M KCl and loaded onto a 3 ml heparin Sepharose column (Amersham Pharmacia) equilibrated in buffer HG+0.5M NaCl (buffer HG=25 mM Hepes-NaOH [pH 7.6], 0.2 mM EDTA, 10% w/v glycerol). After washing with 30 ml of equilibration buffer the column was developed with a 16 ml gradient of 0.5M -1.5M NaCl in buffer HG. Peak fractions were pooled and MES-NaOH [pH 6.1] was added to 50 mM. The NaCl concentration was diluted to 0.5M by addition of buffer MG (25 mM MES-NaOH [pH 6.1], 0.2 mM EDTA, 10% w/v glycerol) and the pooled material loaded onto a 1 ml hydroxylapatite (HAP) column (Bio-Rad, Bio-Gel HTP). The HAP column was washed with 2 ml buffer MG+0.5M NaCl, 3 ml buffer MG+0.5M NaCl+100 mM potassium phosphate [pH 6.1], and 2 ml buffer MG+0.5M NaCl. ResT was then eluted with a 10 ml gradient of 0-0.5M potassium phosphate [pH 6.1] in buffer MG+0.5M NaCl. Peak fractions were collected and adjusted by dialysis to buffer Ac+0.5M NaCl (buffer Ac=25 mM Na acetate [pH 4.5], 0.2 mM EDTA) and loaded onto a second HAP column after clearing the dialysate by centrifugation. This column was washed with 1 ml buffer AcG+0.5M NaCl, 2 ml AcG+0.5M NaCl+150 mM potassium phosphate [pH 6.1], and 3 ml buffer AcG+0.8M NaCl (note that after the load the buffers once again contained 10% w/v glycerol). ResT was eluted with an 8 ml gradient of 0-0.5M potassium phosphate [pH 6.1] in buffer AcG+0.8M NaCl. Peak fractions were pooled and dialyzed against 2 changes of 2L storage buffer (25 mM Tris-HCl [pH 7.8], 0.5M NaCl, 0.2 mM EDTA, 10% w/v glycerol). The purification was monitored by SDS-PAGE and Coomassie blue staining. As evident in FIG. 4, expression of ResT was modest. Yields of purified ResT varied from 0.3 to 0.6 mg per litre of culture, based upon an estimated extinction coefficient of 1 at 280 nm for a concentration of 1 mg/ml.

### Example 4

In vitro Telomere Resolution by ResT

The activity of the purified recombinant protein (ResT) was assayed using plasmids previously established to be in vivo substrates for telomere resolution in B. burgdorferi (Chaconas et al., 2001). pGCL15-6 has a 70 bp replicated left-end telomere from the linear plasmid Ip17 (FIG. 1B). Typical reaction conditions, herein defined as 1× reaction are as follows: 25 mM Tris-HCl [pH 8.5], 100 mM NaCl, 1 mM EDTA, 5 mM spermidine, 100 μg/ml bovine serum albumin, 5 μg/ml substrate DNA and 2 μg/ml ResT. Reactions (typically 30 μl) were incubated at 30° C. for 30 min and terminated by addition of SDS to a final concentration of 0.5%. Spermidine contributes an approximate 3-fold stimulation to the reaction and was therefore omitted from the reactions used for cleavage site determination. Linearized and relaxed plasmid substrates were prepared in 10-20 μg batches by reaction with the specified restriction enzyme with the manufacturers buffers or with Shope Fibroma Virus topoisomerase I (gift of Dr. David Evans, University of Guelph), in ResT reaction buffer. These reactions were then extracted with phenol/chloroform, chloroform alone, and subsequently ethanol precipitated and resuspended in 25 mM Hepes-NaOH [pH 7.6], 0.1 mM EDTA.

The in vitro assay on pGCL15-6 established that it was a highly efficient substrate in vitro for ResT mediated telomere resolution (FIG. 1C, lane 4). The linear reaction product gave rise to the expected 2.0 and 2.6 kb DNA fragments upon cleavage with PstI (lane 5). The linear product also appeared to be terminated by at least one hairpin end because of its snapback properties following heat denaturation (lane 9); removal of the putative hairpin ends with XbaI (lane 7) resulted in a concomitant loss of most of the snap-back. Further analysis of the reaction product is discussed in Example 5.

Telomere resolution by ResT was dependent upon the presence of a functional replicated telomere. The parent vector lacking the telomeric insert was unreactive (FIG. 1C, lane 2). Similar to the in vivo reaction, in vitro telomere resolution was also sequence-specific; a plasmid, (pGCL13-1) carrying a mock replicated telomere (Chaconas et al., 2001), with a 140 bp inverted repeat of the same base composition as the B. burgdorferi telomere, but with an unrelated sequence, was unreactive (data not shown). Additionally, certain point mutations in the replicated telomere were found to abolish the reaction (Yvonne Tourand and G. C.; unpublished results). ResT alone, therefore, appeared to perform an authentic telomere resolution reaction which mimicked the known properties of the in vivo reaction. ResT performed the reaction in a simple buffer without any accessory proteins, divalent metal ions or high energy cofactors. Addition of the B. burgdorferi accessory factors GAC (Knight and Samuels, 1999) or Hbb (Kobryn et al., 2000) did not give detectable stimulation of the reaction (data not shown).

Telomere resolution by ResT was found to be sensitive to the topological state of the substrate plasmid. Relaxed circular, open circular and linearized plasmids were all substrates; however, the supercoiled form of pGCL15-6 was not reactive (FIG. 1C, lane 11). The small amount of product in lane 11 resulted from resolution of the open circular DNA present in the plasmid preparation (nicked monomer, visible on the gel, as well as nicked dimer, which migrated near the top of the gel and is not shown). Further analysis ruled out the possibility that supercoiled pGCL15-6 exposed to ResT had reacted and given rise to a topologically complex product that comigrated with the supercoiled form (data not shown).

### Example 5

Further Analysis of the ResT Reaction Product

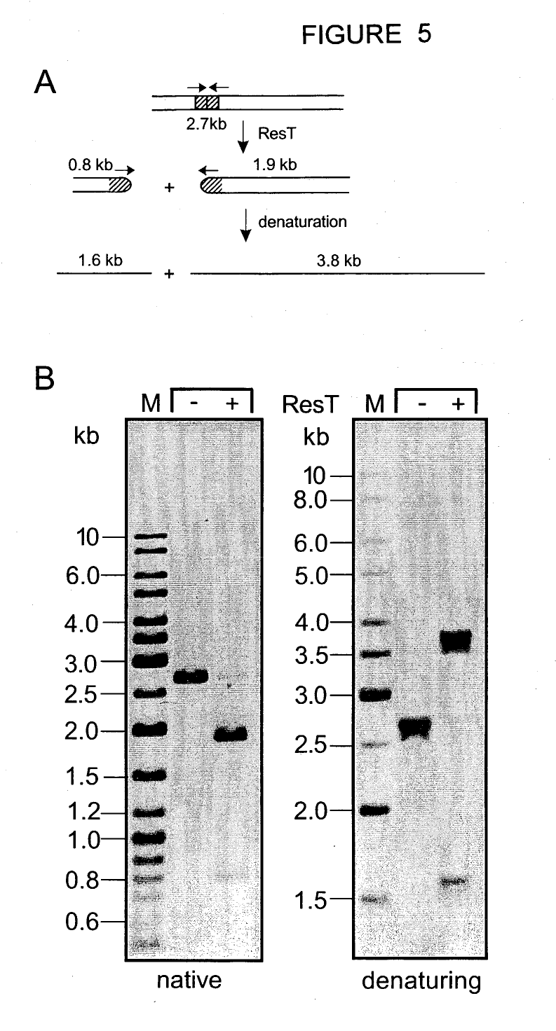

To confirm that ResT generated two hairpins from the reaction with the replicated telomere substrate, resolution products were obtained from a reaction with a pre-linearized 2.7 kb substrate (SspI linearized pGCL47-4, see FIG. 5A). These reaction products were analyzed on native and alkaline agarose gels. In vitro telomere resolution of this substrate resulted in the two expected double stranded fragments of 0.8 and 1.9 kb (FIG. 5B, native gel). In contrast, when the reaction products were examined on a denaturing alkaline agarose gel the DNA migrated as single stranded 1.6 and 3.8 kb species, compared to the single stranded 2.7 kb substrate. These results can only be explained by a covalent linkage between the two DNA strands in each of the ResT products.

Finally, an asymmetrically 3′ end labeled product of the ResT reaction on pGCL47-4 was sequenced by chemical modification followed by strand scission with piperidine (G, A>C & C+T reactions;(Maxam and Gilbert, 1980)). The sequence of the DNA flanking the telomere confirmed that the product was indeed a DNA hairpin. Resolution by ResT did not lead to the loss or change of DNA sequence in the resolved telomere product (data not shown).

### Example 6

Analysis of ResT-DNA Covalent Intermediate

FIG. 6A shows that ResT from B. burgdorferi contains the box A and C motifs (Esposito and Scocca, 1997; Nunes-Duby et al., 1998) common to the tyrosine recombinase family of proteins. This suggested that telomere resolution might proceed through a ResT-DNA covalent intermediate similar to that used in site-specific recombination (Gopaul and Duyne, 1999; Grainge and Jayaram, 1999; Hallet and Sherratt, 1997). We attempted to detect such an intermediate using increased ResT concentrations and suboptimal buffer conditions; this was coupled with short reaction times and termination with 1% SDS, followed by a step to enrich for covalent protein-DNA complexes using potassium precipitation of protein. The reaction products were first examined on an SDS-containing polyacrylamide gel (FIG. 6B). A ResT-dependent protein-DNA complex (P-D) corresponding to about 3% of the total input label was observed using wild type ResT (lane 2). This complex was not observed when ResT_{Y335F}, in which the putative active site tyrosine was changed to phenylalanine, was used (lane 1). This mutant ResT protein did not generate any detectable products under extended reaction times (data not shown). Pronase treatment of the complex observed with wild type ResT released double strand break products (DSB) of the expected size (lane 3), thereby confirming the presence of ResT covalently linked to the DNA substrate. The unequal distribution of the double strand break products released after pronase treatment likely resulted from unequal labeling of the two ends. There is considerable certainty that the protein-DNA complex is a true reaction intermediate for several reasons: 1) It was not observed with ResT_{Y335F}. 2) Renaturation of ResT in the covalent ResT-DNA complex resulted in subsequent intermolecular ligation, indicating that the covalent complex was capable of performing a transesterification reaction (data not shown). 3) A reaction time course displayed early appearance and subsequent decrease of the DNA cleavage intermediate accompanied by accumulation of hairpin product (data not shown).

### Example 7

Preparation of 3′ and 5′ End Labeled Substrates

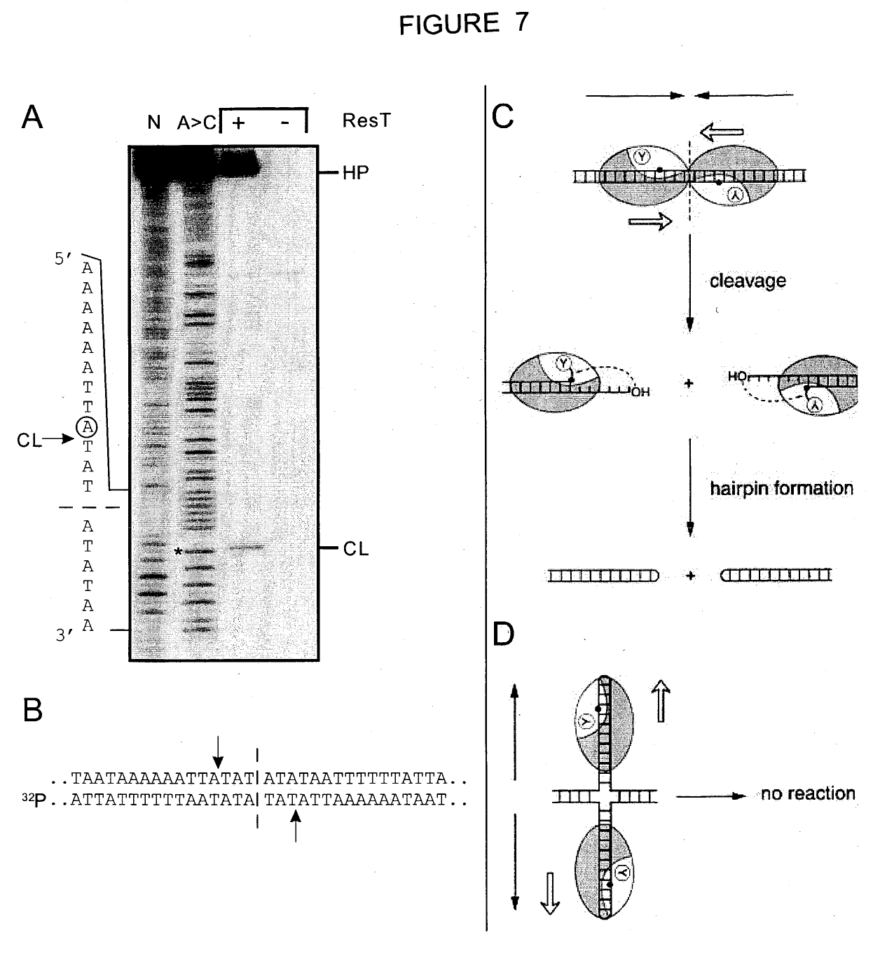

The substrate plasmid pGCL47-4 was cut with NcoI, 3′ end labeled with [α-³²P]dCTP using MMLV reverse transcriptase or 5′ end labeled using [γ-³²P]ATP and T₄ polynucleotide kinase. The label was segregated where indicated, by cleavage with SspI followed by recovery of the desired 0.9 kb fragment from an agarose gel. The labeled fragment (7.5 μg/ml) was used in ResT reactions in 25 mM Tris-HCl [pH 8.5], 100 mM NaCl, 1 mM EDTA, 100 μg/ml bovine serum albumin and 12.6 μg/ml ResT. The reactions were incubated at 30° C. for 30 sec. Aliquots were examined directly on a 7.5% DNA sequencing gel or on a 12% SDS-PAGE gel after potassium precipitation of the SDS treated protein-DNA complex (Trask et al., 1984). The precipitates from this method were desalted by ethanol precipitation and washing. The pellets from the ethanol precipitation were resuspended in buffer containing 0.5% SDS, 50 mM Tris-HCl pH 7.9, 100 mM NaCl, 1 mM DTT and 10 mM MgCl₂. Pronase treatment of the indicated samples was with 2 PUK at 37° C. for 20 min. Production of large amounts of hairpin reaction product for generation of DNA sequencing ladders was produced from 10 μg of 3′ end labeled and segregated NcoI-SspI digested pGCL47-4. Quantitative substrate conversion was aided by long reaction times (4 hours) and by the addition of the stimulatory buffer component ethanediol to 10%. The reaction product was extracted with phenol/chloroform, chloroform alone, and then ethanol precipitated and desalted for Maxam-Gilbert sequencing reactions (Maxam and Gilbert, 1980) to prepare G, A>C and C+T markers for the DNA sequencing gels. The N ladder in FIG. 7A is a mixture of G and C+T reactions that had undergone enough radiolysis in storage to have faint background cleavage at A nucleotides as well.

### Example 8

Analysis of the Covalent Linkage

A 5′ end labeled DNA was therefore used to explore the polarity of attachment using the ResT reaction conditions employed to visualize the covalent complex. The reaction products were analyzed on a DNA sequencing gel (FIG. 6C). A DNA cleavage product was not observed (lane 2) unless the reaction was treated with pronase prior to loading the sequencing gel (lane 3). This indicated a 3′ covalent attachment of ResT on the DNA, likely as a phosphotyrosine linkage.

### Example 9

Determination of the ResT-induced DNA Cleavage Sites in the Replicated Telomere

Having established the polarity of the ResT-DNA linkage, the position of the cleavage site on the replicated telomere was mapped using a 3′ end labeled DNA substrate to generate a protein-free cleavage product (FIG. 7A). The ResT cleavage site was found to lie only three nucleotides away from the axis of symmetry in the inverted repeat. The two ResT cleavage sites are therefore separated by six nucleotides and lie in close proximity on the same side of the double helix. The polarity of cleavage results in 5′ overhangs (FIG. 7B).

### Discussion for Examples 6-9

Reaction Mechanism

The mechanism of action by ResT is a two step transesterification using a protein-DNA intermediate (FIG. 7C), similar to that used by topoisomerases (Wang, 1996) and site-specific recombinases (Hallet and Sherratt, 1997). More specifically, the 3′ polarity of the covalent attachment, apparently through a tyrosine nucleophile, is as previously described for Type IB topoisomerases (Shuman, 1998) and the tyrosine recombinases (Gopaul and Duyne, 1999; Grainge and Jayaram, 1999). The introduction of staggered nicks between six and eight nucleotides apart (six for ResT), the lack of a requirement for divalent metal ions or for a high energy cofactor are features of the reaction for tyrosine recombinases. The link is further substantiated by mutation of the putative active site tyrosine (335) to phenylalanine, which resulted in a catalytically inactive protein.

Sensitivity of Telomere Resolution to Substrate Topology

One of the interesting features of the in vitro telomere resolution reaction catalyzed by ResT is its sensitivity to substrate topology. The reaction is effectively abolished by plasmid supercoiling (FIG. 1C; lane 11). This effect can be readily understood in the context of the very AT-rich inverted repeat structure of the replicated telomere. The free energy of supercoiling in our plasmids causes spontaneous extrusion of the replicated telomere as a stable cruciform (data not shown). This disrupts the axis of symmetry present in the lineform substrate, places the cleavage site far from the strand to which it would eventually be joined and would also block dimerization of ResT (see FIG. 7D). It is also interesting to note that the extruded arms of the DNA cruciform directly mimic the structure of the final telomere products. The inertness of this structure indicates that ResT will not readily run the reverse reaction to reconstitute a replicated telomere under normal reaction conditions.

### Example 10

Inhibition of in vitro ResT Activity and in vivo B. burgdorferi Growth by the Coumarin Antibiotics Coumermycin A1 and Novobiocin

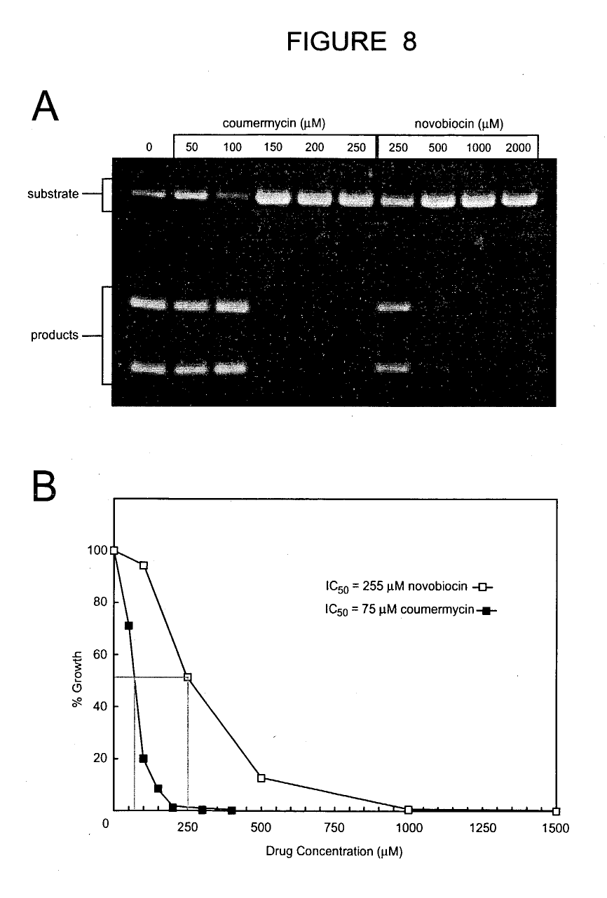

ResT activity was assayed as described above and as shown in FIG. 1, using pYT1 as the substrate. Reactions were performed containing increasing amounts of the topoisomerase inhibitors coumermycin A1 or novobiocin. The reactions were analyzed by agarose gel electrophoresis followed by staining with ethidium bromide. As shown in FIG. 8A, a complete inhibition of ResT activity was observed at 150 μM coumermycin A1 and at 1 mM novobiocin. Other topoisomerase inhibitors (camptothecin (2 mM), NSC-413622 (500 μM), and nalidixic acid (2 mM) did not inhibit ResT (data not shown).

To determine whether coumermycin Al and novobiocin could block growth of B. burgdorferi in culture, a B. burgdorferi strain (NGR) carrying a triple mutation in the gyrB gene (Knight et al., 2000; Rosa et al., 1996) was used (provided by Dr. D. Scott Samuels, University of Montana, Missoula). The triple mutation in the gyrase B subunit renders the mutant DNA gyrase resistant to coumermycin A1 and novobiocin. B. burgdorferi carrying the resistant gyrase was grown in the absence, or in the presence of increasing amounts of coumermycin A1 or novobiocin. FIG. 8B shows that coumermycin A1 effectively inhibited growth of B. burgdorferi carrying the resistant gyrase with an IC₅₀=75 μM. Similarly, novobiocin also effectively inhibited growth of the gyrase mutant with an IC₅₀=255 μM. The drug concentrations for complete inhibition of growth were almost identical to the concentrations of both drugs required for complete inhibition of ResT in vitro.

In summary, the data from Example 10 indicate that the assay for ResT activity can be used to find inhibitors of the enzyme. The data also suggest that ResT inhibitors found using the assay may be potent inhibitors of Borrelia growth and that the assay is therefore a powerful tool for identifying potential growth inhibitors that may be useful for therapeutic or environmental use.

### Example 11

Inhibition of in vitro ResT Activity by Peptide Molecules

In vitro ResT activity was assayed in the presence of peptide inhibitors provided by Dr. Anca Segall, San Diego State University. These peptides were previously characterized as inhibitors of tyrosine recombinase or topoisomerase activity (Cassell et al., 2000; Klemm et al., 2000). The results are summarized in FIG. 9. Peptide #52, which traps the Holliday junction intermediate in λ recombination, did not have any effect upon the reaction. However, several peptides that block the cleavage step of λ integrase, were found to inhibit ResT activity. In addition, peptide #59, which inhibits DNA cleavage by λ integrase, was found to act as a mild stimulator of ResT activity. The results in this example show that some peptide inhibitors of the tyrosine recombinase activity of λ integrase act as ResT inhibitors, demonstrating some similarity between the active sites of these two distinct classes of enzyme. The results also raise the possibility for further development of peptide inhibitors of ResT for therapeutic and/or environmental agents to inhibit growth of Borrelia species.

In summary, the results from FIGS. 8 and 9 support both similarities and yet distinct differences between the active site of ResT, and both the tyrosine recombinases and type 1b topoisomerases. The telomere resolvase, ResT, is a member of a distinct class of enzyme which performs a unique reaction. It does so using an active site which has some similarity to the active sites of both the tyrosine recombinases and type 1b topoisomerases based upon drug inhibition profiles.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

Full Citations for References Referred to in the Specification

Allingham, J. S., Wardle, S. J., and Haniford, D. B., (2001) Determinants for hairpin formation in Tn10 transposition, EMBO J., 20, 2931-2942.

Aravind, L., Makarova, K. S., and Koonin, E. V. (2000). Survey and summary: holliday junction resolvases and related nucleases: identification of new families, phyletic distribution and evolutionary trajectories. Nucleic Acids Res. 28, 3417-3432.

Barbour, A. G. (2001). Borrelia: a diverse and ubiquitous genus of tick-borne pathogens. In Emerging Infections 5, M. W. Scheld, W. A. Craig and J. M. Hughes, eds. (Washington, D.C.: American Society for Microbiology) pp. 153-173.

Barbour, A. G., and Fish, D. (1993). The biological and social phenomenon of Lyme disease. Science 260,1610-1616.

Barbour, A. G., and Garon, C. F. (1987). Linear plasmids of the bacterium Borrelia burgdorferi have covalently closed ends. Science 237, 409-411.

Casjens, S. (1999). Evolution of the linear DNA replicons of the Borrelia spirochetes. Curr. Opin. Microbiol. 2, 529-534.

Casjens, S., Murphy, M., DeLange, M., Sampson, L., van Vugt, R., and Huang, W. M. (1997). Telomeres of the linear chromosomes of Lyme disease spirochaetes: nucleotide sequence and possible exchange with linear plasmid telomeres. Mol. Microbiol. 26, 581-596.

Casjens, S., Palmer, N., Van Vugt, R., Huang, W. H., Stevenson, B., Rosa, P., Lathigra, R., Sutton, G., Peterson, J., Dodson, R. J., Haft, D., Hickey, E., Gwinn, M., White, O., and Fraser, C. M. (2000). A bacterial genome in flux: the twelve linear and nine circular extrachromosomal DNAs in an infectious isolate of the Lyme disease spirochete Borrelia burgdorferi. Mol. Microbiol. 35, 490-516.

Cassell, G., Klemm, M., Pinilla, C., and Segall, A. (2000). Dissection of bacteriophage lambda site-specific recombination using synthetic peptide combinatorial libraries. J. Mol. Biol. 299, 1193-1202.

Chaconas, G., Stewart, P. E., Tilly, K., Bono, J. L., and Rosa, P. (2001). Telomere resolution in the Lyme disease spirochete. EMBO J. 20, 3229-3237.

Davies, D. R., Goryshin, I. Y., Reznikoff, W. S., and Rayment, I., (2000) Three-dimensional structure of the Tn5 synaptic complex transposition intermediate, Science, 289, 77-85.

Deneke, J., Ziegelin, G., Lurz, R., and Lanka, E. (2000). The protelomerase of temperate Escherichia coli phage N15 has cleaving-joining activity. Proc. Natl. Acad. Sci. USA 97, 7721-7726.

Esposito, D., and Scocca, J. J. (1997). The integrase family of tyrosine recombinases: evolution of a conserved active site domain. Nucleic Acids Res. 25, 3605-3614.

Fraser, C. M., Casjens, S., Huang, W. M., Sutton, G. G., Clayton, R., Lathigra, R., White, O., Ketchum, K. A., Dodson, R., Hickey, E. K., Gwinn, M., Dougherty, B., Tomb, J. F., Fleischmann, R. D., Richardson, D., Peterson, J., Kerlavage, A. R., Quackenbush, J., Salzberg, S., Hanson, M., van Vugt, R., Palmer, N., Adams, M. D., Gocayne, J., Weidman, J., Utterback, T., Watthey, L., McDonald, L., Artiach, P., Bowman, C., Garland, S., Fujii, C., Cotton, M. D., Horst, K., Roberts, K., Hatch, B., Smith, H. O., and Venter, J. C. (1997). Genomic sequence of a Lyme disease spirochaete, Borrelia burgdorferi. Nature 390, 580-586.

Gopaul, D. N., and Duyne, G. D. (1999). Structure and mechanism in site-specific recombination. Curr. Opin. Struct. Biol. 9, 14-20.

Grainge, I., and Jayaram, M. (1999). The integrase family of recombinase: organization and function of the active site. Mol. Microbiol. 33, 449-456.

Hallet, B., and Sherratt, D. J. (1997). Transposition and site-specific recombination: adapting DNA cut-and- paste mechanisms to a variety of genetic rearrangements. FEMS Microbiol. Rev. 21, 157-178.

Hinnebusch, J., and Barbour, A. G. (1991). Linear plasmids of Borrelia burgdorferi have a telomeric structure and sequence similar to those of a eukaryotic virus. J. Bacteriol. 173, 7233-7239.

Klemm, M., Cheng, C., Cassell, G., Shuman, S., and Segall, A. M. (2000). Peptide inhibitors of DNA cleavage by tyrosine recombinases and topoisomerases. J. Mol. Biol. 299,1203-1216.

Knight, S. W., and Samuels, D. S. (1999). Natural synthesis of a DNA-binding protein from the C-terminal domain of DNA gyrase A in Borrelia burgdorferi. EMBO J. 18, 4875-4881.

Knight, S. W., Kimmel, B. J., Eggers, C. H., and Samuels, D. S. (2000). Disruption of the Borrelia burgdorferi gac gene, encoding the naturally synthesized GyrA C-terminal domain. J. Bacteriol. 182, 2048-2051.

Kobryn, K., and Chaconas, G. (2001). The circle is broken: Telomere resolution in linear replicons. Current Opinion in Microbiology 4, 558-564.

Kobryn, K., and Chaconas, G. (2002). ResT, a telomere resolvase encoded by the Lyme disease spirochete. Mol. Cell 9,195-201.

Kobryn, K., Naigamwalla, D. Z., and Chaconas, G. (2000). Site-specific DNA binding and bending by the Borrelia burgdorferi Hbb protein. Mol. Microbiol. 37, 145-155.

Lavoie, B. D., and Chaconas, G. (1993). Site-specific HU binding in the Mu transpososome: conversion of a sequence-independent DNA-binding protein into a chemical nuclease. Genes Dev. 7, 2510-2519.

Margolis, N., Hogan, D., Tilly, K., and Rosa, P. A. (1994). Plasmid location of Borrelia purine biosynthesis gene homologs. J. Bacteriol. 176, 6427-6432.

Maxam, A. M., and Gilbert, W. (1980). Sequencing end-labeled DNA with base-specific chemical cleavages. Methods Enzymol. 65, 499-560.

McDowell, J. V., Sung, S. Y., Labandeira-Rey, M., Skare, J. T., and Marconi, R. T. (2001). Analysis of mechanisms associated with loss of infectivity of clonal populations of Borrelia burgdorferi B31MI. Infect. Immun. 69, 3670-3677.

Nunes-Duby, S. E., Kwon, H. J., Tirumalai, R. S., Ellenberger, T., and Landy, A. (1998). Similarities and differences among 105 members of the Int family of site-specific recombinases. Nucleic Acids Res. 26, 391-406.

Picardeau, M., Lobry, J. R., and Hinnebusch, B. J. (2000). Analyzing DNA strand compositional asymmetry to identify candidate replication origins of Borrelia burgdorferi linear and circular plasmids. Genome Res. 10, 1594-1604.

Picardeau, M., Lobry, J. R., and Hinnebusch, B. J. (1999). Physical mapping of an origin of bidirectional replication at the centre of the Borrelia burgdorferi linear chromosome. Mol. Microbiol. 32, 437-445.

Purser, J. E., and Norris, S. J. (2000). Correlation between plasmid content and infectivity in Borrelia burgdorferi. Proc. Natl. Acad. Sci. USA 97, 13865-13870.

Raj, K, Ogston, P. and Beard, P. (2001) Virus-mediated killing of cells that lack p53 activity. Nature, 412, 914-916.

Rezsohazy, R., Hallet, B., Delcour, J., and Mahillon, J., (1993) The IS4 family of insertion sequences: evidence for a conserved transposase motif, Mol. Microbiol., 9, 1283-1295.

Rosa, P., Samuels, D. S., Hogan, D., Stevenson, B., Casjens, S., and Tilly, K. (1996). Directed insertion of a selectable marker into a circular plasmid of Borrelia burgdorferi. J. Bacteriol. 178, 5946-5953.

Rybchin, V. N., and Svarchevsky, A. N. (1999). The plasmid prophage N15: a linear DNA with covalently closed ends. Mol. Microbiol. 33, 895-903.

Sadziene, A., Wilske, B., Ferdows, M. S., and Barbour, A. G. (1993). The cryptic ospC gene of Borrelia burgdorferi B31 is located on a circular plasmid. Infect. Immun. 61, 2192-2195.

Shuman, S. (1998). Vaccinia virus DNA topoisomerase: a model eukaryotic type IB enzyme. Biochim. Biophys. Acta 1400, 321-337.

Tilly, K., Elias, A. F., Errett, J., Fischer, E., Iyer, R., Schwartz, I., Bono, J. L., and Rosa, P. (2001). Genetics and Regulation of Chitobiose Utilization in Borrelia burgdorferi. J. Bacteriol. 183, 5544-5553.

Trask, D. K., DiDonato, J. A., and Muller, M. T. (1984). Rapid detection and isolation of covalent DNA/protein complexes: application to topoisomerase I and II. EMBO J. 3, 671-676.

Vogelstein, B. and Kinzler, K. W. (2001). Achilles' heel of cancer?. Nature, 412, 865.

Wang, J. C. (1996). DNA topoisomerases. Annu. Rev. Biochem. 65, 635-692.

Wang, W., and Malcolm, B. A. (1999). Two-stage PCR protocol allowing introduction of multiple mutations, deletions and insertions using QuikChange Site-Directed Mutagenesis. BioTechniques 26, 680-682.

We claim:

1. A method of identifying a modulator of ResT comprising:
(a) incubating a test substance in the presence of ResT and a telomere resolution substrate; and
(b) determining the effect of the test substance on telomere resolution, wherein a change in telomere resolution as compared to a control means the test substance is a modulator of ResT activity.

2. A method according to claim 1 wherein the telomere resolution substrate comprises a functional replicated telomere.

3. A method according to claim 2 wherein the functional replicated telomere is from a Borrelia species.

4. A method according to claim 3 wherein the replicated telomere comprises at least 38 bp of a replicated telomere.

5. A method according to claim 3 wherein the replicated telomere is pGCL15-6 (SEQ ID NO. 2); pYT1 (SEQ ID NO: 3); pYT10 (SEQ ID NO: 4); or pYT11 (SEQ ID NO: 14).

6. A method according to claim 1 wherein the ResT has the amino acid sequence shown in FIG. 2A (SEQ ID NO: 1) or is an analog, homolog, isoform or fragment of the ResT protein shown in FIG. 2A (SEQ ID NO: 1) that retains the telomere resolvase function.

7. A method according to claim 1 wherein the method comprises:
(a) incubating a test substance in the presence of ResT and a telomere resolution substrate comprising a functional replicated telomere; and
(b) assaying for the presence of an expected product;
wherein a change in an amount of expected product in the presence of the test substance compared to a control indicates that the test substance is a modulator of ResT.

8. A method according to claim 7 further comprising treating the expected product with a restriction enzyme to provide one or more DNA fragments having known sizes and assaying for the DNA fragments.

9. A method according to claim 8 wherein the telomere resolution substrate is a circular plasmid comprising SEQ ID NO: 2, the restriction enzyme is PstI, the resulting DNA fragments are 2.0 and 2.6 kb in size.

10. A method according to claim 1 further comprising adding spermidine.

11. A method of modulating telomere resolution comprising administering an effective amount of a modulator of ResT to a cell or animal in need thereof.

12. A method of inhibiting; DNA replication comprising administering an effective amount of a modulator of ResT to a cell or animal in need thereof.

13. A method according to claim 11 wherein the modulator of ResT is identified according to the method of any one of claims 1-10.

14. A method according to claim 11 wherein the modulator of ResT is an antibody that binds to ResT.

15. A method according to claim 11 wherein the modulator of ResT is an antisense oligonucleotide that is complementary to a nucleic acid sequence encoding ResT.

16. A method according to claim 11 wherein the modulator of ResT is a peptide mimetic.

17. A method according to claim 11 wherein the modulator of ResT is a coumarin antibiotic.

18. A method according to claim 17 wherein the coumarin antibiotic ResT is coumermycin A1 or novobiocin.

19. A method according to claim 11 wherein the modulator of ResT is a peptide having the sequence WRRCRW (SEQ ID NO: 24); WRRWCR (SEQ ID NO: 25); WRYRCR (SEQ ID NO: 26); RCCYWW (SEQ ID NO: 28) or WRWYCRCK (SEQ ID NO: 31) as is shown in FIG. 9.

20. A method of treating or preventing Borrelia infection comprising administering an effective amount of an inhibitor of ResT to an animal in need thereof.

21. A method according to claim 20 wherein the inhibitor is identified according to the screening assay according to any one of claims 1 to 10.

22. A method according to claim 20 wherein the inhibitor of ResT is an antibody that binds to ResT.

23. A method according to claim 20 wherein the inhibitor of ResT is an antisense oligonucleotide that is complementary to either a nucleic acid sequence encoding ResT, or a nucleic acid sequence which is part of the ResT DNA substrate.

24. A method according to claim 20 wherein the inhibitor of ResT is a peptide mimetic.

25. A method according to claim 20 wherein the modulator of ResT is a coumarin antibiotic.

26. A method according to claim 25 wherein the coumarin antibiotic is coumermycin A1 or novobiocin.

27. A method according to claim 20 wherein the modulator of ResT is a peptide having the sequence WRRCRW (SEQ ID NO: 24); WRRWCR (SEQ ID NO: 25); WRYRCR (SEQ ID NO: 26); RCCYWW (SEQ ID NO: 28) or WRWYCRCK (SEQ ID NO: 31) as is shown in FIG. 9.

28. A method according to claim 20 wherein the animal is a human.

29. A method according to claim 20 wherein the animal is a tick, lice, mouse, bird or lizard.

30. A method of treating or preventing a poxviral infection comprising administering an effective amount of an inhibitor of ResT to an animal in need thereof.

31. A method according to claim 30 wherein the poxviral infection is smallpox.

32. A kit for use in identifying a modulator of ResT comprising an aliquot of ResT and an aliquot of a telomere resolution substrate.

33. A kit according to claim 32 wherein the telomere resolution substrate comprises a functional replicated telomere.

34. A kit according to claim 33 wherein the functional replicated telomere is from a Borrelia species.

35. A kit according to claim 33 wherein the replicated telomere comprises at least 38 bp of a replicated telomere.

36. A kit according to claim 33 wherein the replicated telomere is pGCL15-6 (SEQ ID NO. 2); pYT1 (SEQ ID NO: 3); pYT10 (SEQ ID NO: 4); or pYT11 (SEQ ID NO: 14).

37. A kit according to claim 32 wherein the ResT has the amino acid sequence shown in FIG. 2A (SEQ ID NO: 1) or is an analog, homolog, isoform or fragment of the ResT protein shown in FIG. 2A (SEQ ID NO: 1) that retains the telomerase resolvase function.

38. A kit according to claim 32 further comprising a restriction enzyme.

39. A kit according to claim 38 wherein the restriction enzyme is PstI.

40. A kit according to claim 33 further comprising spermidine, or any other stimulating agent.

41. A method of preparing a pharmaceutical composition for use in modulating the activity of ResT comprising mixing a modulator of ResT with a suitable diluent or carrier.

42. A method of preparing a pharmaceutical composition for use in modulating the activity of ResT comprising mixing a modulator of ResT with a suitable diluent or carrier wherein the modulator of ResT is identified according to the method of claim 1.

43. A method according to claim 41 wherein the modulator of ResT is a coumarin antibiotic.

44. A method according to claim 43 wherein the coumarin antibiotic is coumermycin A1 or novobiocin.

45. A method according to claim 41 wherein the modulator of ResT is a peptide having the sequence WRRCRW (SEQ ID NO: 24); WRRWCR (SEQ ID NO: 25); WRYRCR (SEQ ID NO: 26); RCCYWW (SEQ ID NO: 28) or WRWYCRCK (SEQ ID NO: 31) as is shown in FIG. 9.

46. An isolated ResT protein having the amino acid sequence shown in FIG. 2A (SEQ ID NO: 1) or an analog, homolog, isoform or fragment of the protein shown in FIG. 2A (SEQ ID NO: 1) that retains the telomere resolvase function.

47. A use of a ResT protein according to claim 46 as a telomere resolvase.
